SEQUENCE LISTING (250 sequences)

SEQ ID NO: 1 - protein, 539 aa, synthetic construct
  REGION 1..539: note = PIV191469
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGSWHQSSTTIIVILIMMIILFIINITIITIAIKYYRIQKRDRVDQNDKPYVLTNK

SEQ ID NO: 2 - protein, 491 aa, synthetic construct
  REGION 1..491: note = PIV190058
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGGSEPEA

SEQ ID NO: 3 - protein, 23 aa, synthetic construct
  REGION 1..23: note = GCN4
EDKIEEILSKIYHIENEIARIKK

SEQ ID NO: 4 - protein, 516 aa, synthetic construct
  REGION 1..516: note = PIV171432
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 5 - protein, 491 aa, synthetic construct
  REGION 1..491: note = PIV190059
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIGGSEPEA

SEQ ID NO: 6 - protein, 516 aa, synthetic construct
  REGION 1..516: note = PIV191486
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 7 - protein, 516 aa, synthetic construct
  REGION 1..516: note = PIV191489
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLLLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 8 - protein, 516 aa, synthetic construct
  REGION 1..516: note = PIV191501
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 9 - protein, 491 aa, synthetic construct
  REGION 1..491: note = PIV191523
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDVSIELNKVKSDLEEVKEWLRRVNQKLDSIGSGEPEA

SEQ ID NO: 10 - protein, 491 aa, synthetic construct
  REGION 1..491: note = PIV191528
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDVSIELNKVKSKLEDVKEWLRRVNQKLDSIGSGEPEA

SEQ ID NO: 11 - protein, 491 aa, synthetic construct
  REGION 1..491: note = PIV191530
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDLEEVKEWLRRVNQKLDSIGSGEPEA

SEQ ID NO: 12 - protein, 491 aa, synthetic construct
  REGION 1..491: note = PIV191531
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKIKSDLEEIKEWLRRVNQKLDSIGSGEPEA

SEQ ID NO: 13 - protein, 516 aa, synthetic construct
  REGION 1..516: note = PIV191551
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 14 - protein, 516 aa, synthetic construct
  REGION 1..516: note = PIV191557
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 15 - protein, 516 aa, synthetic construct
  REGION 1..516: note = PIV191560
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 16 - protein, 516 aa, synthetic construct
  REGION 1..516: note = PIV191561
MPISILLIITTMIMASHCQIDITKLQHVGVLVNSPKGMKISQNFETRYLILSLIPKIEDSNSCGDQQIKQYKRLLDRLIIPLYDCLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSCQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 17 - protein, 516 aa, synthetic construct
  REGION 1..516: note = PIV191580
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 18 - protein, 491 aa, synthetic construct
  REGION 1..491: note = PIV191593
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGSGEPEA

SEQ ID NO: 19 - protein, 491 aa, synthetic construct
  REGION 1..491: note = PIV191594
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIGSGEPEA

SEQ ID NO: 20 - protein, 516 aa, synthetic construct
  REGION 1..516: note = PIV200253
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 21 - protein, 516 aa, synthetic construct
  REGION 1..516: note = PIV200254
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 22 - protein, 516 aa, synthetic construct
  REGION 1..516: note = PIV200255
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLPFYTPDDITLNNSVALNPIDISIELNKAKSDLEESKEWIRRSNQKLDSIEDKIEEILSKIYHIENEIARIKKLIGEAEPEA

SEQ ID NO: 23 - protein, 516 aa, synthetic construct
  REGION 1..516: note = PIV200256
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 24 - protein, 516 aa, synthetic construct
  REGION 1..516: note = PIV200257
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTLVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 25 - protein, 516 aa, synthetic construct
  REGION 1..516: note = PIV200258
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 26 - protein, 516 aa, synthetic construct
  REGION 1..516: note = PIV200259
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLLLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 27 - protein, 516 aa, synthetic construct
  REGION 1..516: note = PIV200260
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALDQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 28 - protein, 516 aa, synthetic construct
  REGION 1..516: note = PIV200261
MPISILLIITTMIMASHCQIDITKLQHVGVLVNSPKGCKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNCQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 29 - protein, 516 aa, synthetic construct
  REGION 1..516: note = PIV200262
MPISILLIITTMIMASHCQIDITKLQHVGVLVNSPKGCKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNCQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 30 - protein, 516 aa, synthetic construct
  REGION 1..516: note = PIV200263
MPISILLIITTMIMASHCQIDITKLQHVGVLVNSPKGCKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNCQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 31 - protein, 516 aa, synthetic construct
  REGION 1..516: note = PIV200264
MPISILLIITTMIMASHCQIDITKLQHVGVLVNSPKGCKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNCQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLPFYTPDDITLNNSVALNPIDISIELNKAKSDLEESKEWIRRSNQKLDSIEDKIEEILSKIYHIENEIARIKKLIGEAEPEA

SEQ ID NO: 32 - protein, 516 aa, synthetic construct
  REGION 1..516: note = PIV200265
MPISILLIITTMIMASHCQIDITKLQHVGVLVNSPKGCKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNCQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 33 - protein, 516 aa, synthetic construct
  REGION 1..516: note = PIV200266
MPISILLIITTMIMASHCQIDITKLQHVGVLVNSPKGCKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNCQIYKVDSISYNIQNREWYIPLPSHIMTKGAFLGGADVKECIEAFSSYICPSDPGFVLNHEMESCLSGNISQCPRTLVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 34 - protein, 516 aa, synthetic construct
  REGION 1..516: note = PIV200267
MPISILLIITTMIMASHCQIDITKLQHVGVLVNSPKGCKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNCQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 35 - protein, 516 aa, synthetic construct
  REGION 1..516: note = PIV200268
MPISILLIITTMIMASHCQIDITKLQHVGVLVNSPKGCKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLLLGIALTQHYSELTNIFGDNIGSLQEKGIKLQGIASLYRTNITEIFTTSTVDKYDIYDLLFTESIKVRVIDVDLNDYSITLQVRLPLLTRLLNCQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 36 - protein, 516 aa, synthetic construct
  REGION 1..516: note = PIV200269
MPISILLIITTMIMASHCQIDITKLQHVGVLVNSPKGCKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALDQHYSELTNIFGDNIGSLQEKGIKLQGIASLYRTNITEIFTTSTVDKYDIYDLLFTESIKVRVIDVDLNDYSITLQVRLPLLTRLLNCQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 37 - protein, 516 aa, synthetic construct
  REGION 1..516: note = PIV200270
MPISILLIITTMIMASHCQIDITKLQHVGVLVNSPKGMKIPQNFETRYLILSLIPKIEDSNSCGDQQIKQYKRLLDRLIIPLYDCLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSCQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 38 - protein, 516 aa, synthetic construct
  REGION 1..516: note = PIV200271
MPISILLIITTMIMASHCQIDITKLQHVGVLVNSPKGCKISQNFETRYLILSLIPKIEDSNSCGDQQIKQYKRLLDRLIIPLYDCLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSCQEKGIKLQGIASLYRTNITEIFTTSTVDKYDIYDLLFTESIKVRVIDVDLNDYSITLQVRLPLLTRLLNCQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 39 - protein, 516 aa, synthetic construct
  REGION 1..516: note = PIV200272
MPISILLIITTMIMASHCQIDITKLQHVGVLVNSPKGMKISQNFETRYLILSLIPKIEDSNSCGDQQIKQYKRLLDRLIIPLYDCLRLQKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLQLGIALTQHYSELTNIFGDNIGSCQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 40 - protein, 516 aa, synthetic construct
  REGION 1..516: note = PIV200273
MPISILLIITTMIMASHCQIDITKLQHVGVLVNSPKGMKISQNFETRYLILSLIPKIEDSNSCGDQQIKQYKRLLDRLIIPLYDCLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSCQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 41 - protein, 516 aa, synthetic construct
  REGION 1..516: note = PIV200274
MPISILLIITTMIMASHCQIDITKLQHVGVLVNSPKGMKISQNFETRYLILSLIPKIEDSNSCGDQQIKQYKRLLDRLIIPLYDCLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSCQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLPFYTPDDITLNNSVALNPIDISIELNKAKSDLEESKEWIRRSNQKLDSIEDKIEEILSKIYHIENEIARIKKLIGEAEPEA

SEQ ID NO: 42 - protein, 516 aa, synthetic construct
  REGION 1..516: note = PIV200275
MPISILLIITTMIMASHCQIDITKLQHVGVLVNSPKGMKISQNFETRYLILSLIPKIEDSNSCGDQQIKQYKRLLDRLIIPLYDCLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSCQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 43 - protein, 516 aa, synthetic construct
  REGION 1..516: note = PIV200276
MPISILLIITTMIMASHCQIDITKLQHVGVLVNSPKGMKISQNFETRYLILSLIPKIEDSNSCGDQQIKQYKRLLDRLIIPLYDCLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSCQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTLVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 44 - protein, 516 aa, synthetic construct
  REGION 1..516: note = PIV200277
MPISILLIITTMIMASHCQIDITKLQHVGVLVNSPKGMKISQNFETRYLILSLIPKIEDSNSCGDQQIKQYKRLLDRLIIPLYDCLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPSIARLGCEAAGLQLGIALTQHYSELTNIFGDNIGSCQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 45 - protein, 516 aa, synthetic construct
  REGION 1..516: note = PIV200278
MPISILLIITTMIMASHCQIDITKLQHVGVLVNSPKGMKISQNFETRYLILSLIPKIEDSNSCGDQQIKQYKRLLDRLIIPLYDCLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLLLGIALTQHYSELTNIFGDNIGSCQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 46 - protein, 516 aa, synthetic construct
  REGION 1..516: note = PIV200279
MPISILLIITTMIMASHCQIDITKLQHVGVLVNSPKGMKISQNFETRYLILSLIPKIEDSNSCGDQQIKQYKRLLDRLIIPLYDCLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALDQHYSELTNIFGDNIGSCQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 47 - protein, 516 aa, synthetic construct
  REGION 1..516: note = PIV200280
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 48 - protein, 516 aa, synthetic construct
  REGION 1..516: note = PIV200281
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLPFYTPDDITLNNSVALNPIDISIELNKAKSDLEESKEWIRRSNQKLDSIEDKIEEILSKIYHIENEIARIKKLIGEAEPEA

SEQ ID NO: 49 - protein, 516 aa, synthetic construct
  REGION 1..516: note = PIV200282
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 50 - protein, 516 aa, synthetic construct
  REGION 1..516: note = PIV200283
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTLVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 51 - protein, 516 aa, synthetic construct
  REGION 1..516: note = PIV200284
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGPLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 52 - protein, 516 aa, synthetic construct
  REGION 1..516: note = PIV200285
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLLLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 53 - protein, 516 aa, synthetic construct
  REGION 1..516: note = PIV200286
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLQLGIALDQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 54 - protein, 516 aa, synthetic construct
  REGION 1..516: note = PIV200287
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLPFYTPDDITLNNSVALNPIDISIELNKAKSDLEESKEWIRRSNQKLDSIEDKIEEILSKIYHIENEIARIKKLIGEAEPEA

SEQ ID NO: 55 - protein, 516 aa, synthetic construct
  REGION 1..516: note = PIV200288
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 56 - protein, 516 aa, synthetic construct
  REGION 1..516: note = PIV200289
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTLVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 57 - protein, 516 aa, synthetic construct
  REGION 1..516: note = PIV200290
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 58 - protein, 516 aa, synthetic construct
  REGION 1..516: note = PIV200291
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLLLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 59 - protein, 516 aa, synthetic construct
  REGION 1..516: note = PIV200292
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALDQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 60 - protein, 516 aa, synthetic construct
  REGION 1..516: note = PIV200293
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLPFYTPDDITLNNSVALNPIDISIELNKAKSDLEESKEWIRRSNQKLDSIEDKIEEILSKIYHIENEIARIKKLIGEAEPEA

SEQ ID NO: 61 - protein, 516 aa, synthetic construct
  REGION 1..516: note = PIV200294
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTLVTSDIVPRYAFVNGGVVANCITTTCTCNGIGNRINQPPDQGVKIITHKECNTIGINGMLFNTNKEGTLPFYTPDDITLNNSVALNPIDISIELNKAKSDLEESKEWIRRSNQKLDSIEDKIEEILSKIYHIENEIARIKKLIGEAEPEA

SEQ ID NO: 62 - protein, 516 aa, synthetic construct
  REGION 1..516: note = PIV200295
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLPFYTPDDITLNNSVALNPIDISIELNKAKSDLEESKEWIRRSNQKLDSIEDKIEEILSKIYHIENEIARIKKLIGEAEPEA

SEQ ID NO: 63 - protein, 516 aa, synthetic construct
  REGION 1..516: note = PIV200296
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLLLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLPFYTPDDITLNNSVALNPIDISIELNKAKSDLEESKEWIRRSNQKLDSIEDKIEEILSKIYHIENEIARIKKLIGEAEPEA

SEQ ID NO: 64 - protein, 516 aa, synthetic construct
  REGION 1..516: note = PIV200297
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALDQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLPFYTPDDITLNNSVALNPIDISIELNKAKSDLEESKEWIRRSNQKLDSIEDKIEEILSKIYHIENEIARIKKLIGEAEPEA

SEQ ID NO: 65 - protein, 516 aa, synthetic construct
  REGION 1..516: note = PIV200298
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTLVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 66 - protein, 516 aa, synthetic construct
  REGION 1..516: note = PIV200299
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 67 - protein, 516 aa, synthetic construct
  REGION 1..516: note = PIV200300
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLLLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 68 - protein, 516 aa, synthetic construct
  REGION 1..516: note = PIV200301
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALDQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 69 - protein, 516 aa, synthetic construct
  REGION 1..516: note = PIV200302
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTLVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 70 - protein, 516 aa, synthetic construct
  REGION 1..516: note = PIV200303
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLLLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTLVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 71 - protein, 516 aa, synthetic construct
  REGION 1..516: note = PIV200304
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALDQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTLVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 72 - protein, 516 aa, synthetic construct
  REGION 1..516: note = PIV200305
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 73 - protein, 516 aa, synthetic construct
  REGION 1..516: note = PIV200306
MPISILLIITTMIMASHCQIDITKLQHVGVLVNSPKGCKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNCQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 74 - protein, 516 aa, synthetic construct
  REGION 1..516: note = PIV200307
MPISILLIITTMIMASHCQIDITKLQHVGVLVNSPKGMKISQNFETRYLILSLIPKIEDSNSCGDQQIKQYKRLLDRLIIPLYDCLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSCIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 75 - protein, 516 aa, synthetic construct
  REGION 1..516: note = PIV200308
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 76 - protein, 516 aa, synthetic construct
  REGION 1..516: note = PIV200309
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 77 - protein, 516 aa, synthetic construct
  REGION 1..516: note = PIV200310
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLPFYTPDDITLNNSVALNPIDISIELNKAKSDLEESKEWIRRSNQKLDSIEDKIEEILSKIYHIENEIARIKKLIGEAEPEA

SEQ ID NO: 78 - protein, 516 aa, synthetic construct
  REGION 1..516: note = PIV200311
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 79 - protein, 516 aa, synthetic construct
  REGION 1..516: note = PIV200312
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTLVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 80 - protein, 516 aa, synthetic construct
  REGION 1..516: note = PIV200313
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSPGNLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 81 - protein, 516 aa, synthetic construct
  REGION 1..516: note = PIV200314
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 82 - protein, 516 aa, synthetic construct
  REGION 1..516: note = PIV200315
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 83 - protein, 516 aa, synthetic construct
  REGION 1..516: note = PIV200316
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 84 - protein, 516 aa, synthetic construct
  REGION 1..516: note = PIV200317
MPISILLIITTMIMASHCQIDITKLQHVGVLVNSPKGCKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNCQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 85 - protein, 516 aa, synthetic construct
  REGION 1..516: note = PIV200318
MPISILLIITTMIMASHCQIDITKLQHVGVLVNSPKGMKISQNFETRYLILSLIPKIEDSNSCGDQQIKQYKRLLDRLIIPLYDCLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPCIARLGCEACGLQLGIALTQHYSELTNIFGDNIGSCQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 86 - protein, 516 aa, synthetic construct
  REGION 1..516: note = PIV200319
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 87 - protein, 516 aa, synthetic construct
  REGION 1..516: note = PIV200320
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 88 - protein, 516 aa, synthetic construct
  REGION 1..516: note = PIV200321
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLPFYTPDDITLNNSVALNPIDISIELNKAKSDLEESKEWIRRSNQKLDSIEDKIEEILSKIYHIENEIARIKKLIGEAEPEA

SEQ ID NO: 89 - protein, 516 aa, synthetic construct
  REGION 1..516: note = PIV200322
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 90 - protein, 516 aa, synthetic construct
  REGION 1..516: note = PIV200323
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTLVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 91 - protein, 516 aa, synthetic construct
  REGION 1..516: note = PIV200324
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPCIARLGCEACGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 92 - protein, 516 aa, synthetic construct; identical to SEQ ID NO: 6
  REGION 1..516: note = PIV200325

SEQ ID NO: 93 - protein, 516 aa, synthetic construct
  REGION 1..516: note = PIV200326
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPCIARLGCEACGLLLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 94 - protein, 516 aa, synthetic construct
  REGION 1..516: note = PIV200327
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPCIARLGCEACGLQLGIALDQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 95 - protein, 516 aa, synthetic construct
  REGION 1..516: note = PIV200328
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPSIARLGCEAAGLLLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 96 - protein, 516 aa, synthetic construct
  REGION 1..516: note = PIV200329
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPSIARLGCEAAGLQLGIALDQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 97 - protein, 516 aa, synthetic construct
  REGION 1..516: note = PIV200330
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLLLGIALDQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 98 - protein, 516 aa, synthetic construct
  REGION 1..516: note = PIV200721
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 99 - protein, 516 aa, synthetic construct
  REGION 1..516: note = PIV200722
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 100 - protein, 516 aa, synthetic construct
  REGION 1..516: note = PIV200723
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLLLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 101 - protein, 516 aa, synthetic construct
  REGION 1..516: note = PIV200724
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALDQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 102 - protein, 516 aa, synthetic construct
  REGION 1..516: note = PIV200878
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 103 - protein, 516 aa, synthetic construct
  REGION 1..516: note = PIV200879
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 104 - protein, 516 aa, synthetic construct
  REGION 1..516: note = PIV200880
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 105 - protein, 516 aa, synthetic construct
  REGION 1..516: note = PIV200881
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 106 - protein, 516 aa, synthetic construct
  REGION 1..516: note = PIV200882
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 107 - protein, 516 aa, synthetic construct
  REGION 1..516: note = PIV200883
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 108 - protein, 516 aa, synthetic construct
  REGION 1..516: note = PIV200884
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 109 - protein, 516 aa, synthetic construct
  REGION 1..516: note = PIV200885
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 110 - protein, 516 aa, synthetic construct
  REGION 1..516: note = PIV200886
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 111 - protein, 516 aa, synthetic construct
  REGION 1..516: note = PIV200887
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 112 - protein, 516 aa, synthetic construct
  REGION 1..516: note = PIV200888
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 113 - protein, 516 aa, synthetic construct
  REGION 1..516: note = PIV200889
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 114 - protein, 516 aa, synthetic construct
  REGION 1..516: note = PIV200890
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 115 - protein, 516 aa, synthetic construct
  REGION 1..516: note = PIV200891
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 116 - protein, 516 aa, synthetic construct
  REGION 1..516: note = PIV200892
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 117 - protein, 516 aa, synthetic construct
  REGION 1..516: note = PIV200893
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 118 - protein, 516 aa, synthetic construct
  REGION 1..516: note = PIV200894
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 119 - protein, 516 aa, synthetic construct
  REGION 1..516: note = PIV200895
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 120 - protein, 516 aa, synthetic construct
  REGION 1..516: note = PIV200896
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 121 - protein, 516 aa, synthetic construct
  REGION 1..516: note = PIV200897
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 122 - protein, 516 aa, synthetic construct
  REGION 1..516: note = PIV200898
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 123 - protein, 516 aa, synthetic construct
  REGION 1..516: note = PIV200899
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 124 - protein, 516 aa, synthetic construct
  REGION 1..516: note = PIV200900
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 125 - protein, 516 aa, synthetic construct
  REGION 1..516: note = PIV200901
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 126 - protein, 516 aa, synthetic construct
  REGION 1..516: note = PIV200902
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 127 - protein, 516 aa, synthetic construct
  REGION 1..516: note = PIV200903
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 128 - protein, 516 aa, synthetic construct
  REGION 1..516: note = PIV200904
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 129 - protein, 516 aa, synthetic construct
  REGION 1..516: note = PIV200905
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 130 - protein, 516 aa, synthetic construct
  REGION 1..516: note = PIV200906
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 131 - protein, 516 aa, synthetic construct
  REGION 1..516: note = PIV200907
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 132 - protein, 516 aa, synthetic construct
  REGION 1..516: note = PIV200908
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 133 - protein, 516 aa, synthetic construct
  REGION 1..516: note = PIV200909
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 134 - protein, 516 aa, synthetic construct
  REGION 1..516: note = PIV200910
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 135 - protein, 516 aa, synthetic construct
  REGION 1..516: note = PIV200911
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 136 - protein, 516 aa, synthetic construct
  REGION 1..516: note = PIV200912
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 137 - protein, 516 aa, synthetic construct
  REGION 1..516: note = PIV200913
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 138 - protein, 516 aa, synthetic construct
  REGION 1..516: note = PIV200914
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 139 - protein, 516 aa, synthetic construct
  REGION 1..516: note = PIV200915
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 140 - protein, 516 aa, synthetic construct
  REGION 1..516: note = PIV200916
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 141 - protein, 516 aa, synthetic construct
  REGION 1..516: note = PIV200917
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 142 - protein, 516 aa, synthetic construct
  REGION 1..516: note = PIV200918
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 143 - protein, 516 aa, synthetic construct
  REGION 1..516: note = PIV200919
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 144 - protein, 516 aa, synthetic construct
  REGION 1..516: note = PIV200920
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 145 - protein, 516 aa, synthetic construct
  REGION 1..516: note = PIV200921
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 146 - protein, 516 aa, synthetic construct
  REGION 1..516: note = PIV200922
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 147 - protein, 516 aa, synthetic construct
  REGION 1..516: note = PIV200923
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 148 - protein, 516 aa, synthetic construct
  REGION 1..516: note = PIV200924
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 149 - protein, 516 aa, synthetic construct
  REGION 1..516: note = PIV200925
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 150 - protein, 516 aa, synthetic construct
  REGION 1..516: note = PIV200926
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 151 - protein, 516 aa, synthetic construct
  REGION 1..516: note = PIV200927
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 152 - protein, 516 aa, synthetic construct
  REGION 1..516: note = PIV200928
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 153 - protein, 516 aa, synthetic construct
  REGION 1..516: note = PIV200929
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 154 - protein, 516 aa, synthetic construct
  REGION 1..516: note = PIV200930
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 155 - protein, 516 aa, synthetic construct
  REGION 1..516: note = PIV200931
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 156 - protein, 516 aa, synthetic construct
  REGION 1..516: note = PIV200932
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 157 - protein, 516 aa, synthetic construct
  REGION 1..516: note = PIV200933
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 158 - protein, 516 aa, synthetic construct
  REGION 1..516: note = PIV200934
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 159 - protein, 516 aa, synthetic construct
  REGION 1..516: note = PIV200935
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 160 - protein, 516 aa, synthetic construct
  REGION 1..516: note = PIV200936
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLLLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 161 - protein, 516 aa, synthetic construct
  REGION 1..516: note = PIV200937
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLQLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 162 - protein, 516 aa, synthetic construct
  REGION 1..516: note = PIV200938
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPCIARLGCEACGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 163 - protein, 516 aa, synthetic construct
  REGION 1..516: note = PIV200939
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 164 - protein, 516 aa, synthetic construct
  REGION 1..516: note = PIV200940
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPCIARLGCEACGLLLGIALDQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 165 - protein, 491 aa, synthetic construct
  REGION 1..491: note = PIV200941
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIGSGEPEA

SEQ ID NO: 166 - protein, 491 aa, synthetic construct
  REGION 1..491: note = PIV200943
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWIRRSNQKLDSIGSGEPEA

SEQ ID NO: 167 - protein, 491 aa, synthetic construct
  REGION 1..491: note = PIV200945
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRVNQKLDSIGSGEPEA

SEQ ID NO: 168 - protein, 491 aa, synthetic construct
  REGION 1..491: note = PIV200947
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDLEEVKEWIRRSNQKLDSIGSGEPEA

SEQ ID NO: 169 - protein, 491 aa, synthetic construct
  REGION 1..491: note = PIV200949
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDLEESKEWIRRVNQKLDSIGSGEPEA

SEQ ID NO: 170 - protein, 491 aa, synthetic construct
  REGION 1..491: note = PIV200950
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWLRRSNQKLDSIGSGEPEA

SEQ ID NO: 171 - protein, 491 aa, synthetic construct
  REGION 1..491: note = PIV200951
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWIRRVNQKLDSIGSGEPEA

SEQ ID NO: 172 - protein, 491 aa, synthetic construct
  REGION 1..491: note = PIV200952
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWLRRVNQKLDSIGSGEPEA

SEQ ID NO: 173 - protein, 491 aa, synthetic construct
  REGION 1..491: note = PIV200953
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDLEEVKEWLRRSNQKLDSIGSGEPEA

SEQ ID NO: 174 - protein, 491 aa, synthetic construct
  REGION 1..491: note = PIV200954
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDLEEVKEWIRRVNQKLDSIGSGEPEA

SEQ ID NO: 175 - protein, 491 aa, synthetic construct
  REGION 1..491: note = PIV200955
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDLEESKEWLRRVNQKLDSIGSGEPEA

SEQ ID NO: 176 - protein, 491 aa, synthetic construct
  REGION 1..491: note = PIV200956
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWLRRVNQKLDSIGSGEPEA

SEQ ID NO: 177 - protein, 491 aa, synthetic construct
  REGION 1..491: note = PIV200957
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDLEEVKEWLRRVNQKLDSIGSGEPEA

SEQ ID NO: 178 - protein, 491 aa, synthetic construct
  REGION 1..491: note = PIV200958
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDFEEVKEWLRRVNQKLDSIGSGEPEA

SEQ ID NO: 179 - protein, 491 aa, synthetic construct
  REGION 1..491: note = PIV200960
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRSNQKLDSIGGSEPEA

SEQ ID NO: 180 - protein, 491 aa, synthetic construct
  REGION 1..491: note = PIV200962
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRVNQKLDSIGGSEPEA

SEQ ID NO: 181 - protein, 491 aa, synthetic construct
  REGION 1..491: note = PIV200964
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKVKSDLEEVKEWLRRVNQKLDSIGGSEPEA

SEQ ID NO: 182 - protein, 491 aa, synthetic construct
  REGION 1..491: note = PIV200965
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKVKSDFEEVKEWLRRVNQKLDSIGGSEPEA

SEQ ID NO: 183 - protein, 491 aa, synthetic construct
  REGION 1..491: note = PIV200967
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWIRRSNQKLDSIGSGEPEA

SEQ ID NO: 184 - protein, 491 aa, synthetic construct
  REGION 1..491: note = PIV200969
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRVNQKLDSIGSGEPEA

SEQ ID NO: 185 - protein, 491 aa, synthetic construct
  REGION 1..491: note = PIV200971
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDLEEVKEWIRRSNQKLDSIGSGEPEA

SEQ ID NO: 186 - protein, 491 aa, synthetic construct
  REGION 1..491: note = PIV200973
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDLEESKEWIRRVNQKLDSIGSGEPEA

SEQ ID NO: 187 - protein, 491 aa, synthetic construct
  REGION 1..491: note = PIV200974
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWLRRSNQKLDSIGSGEPEA

SEQ ID NO: 188 - protein, 491 aa, synthetic construct
  REGION 1..491: note = PIV200975
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWIRRVNQKLDSIGSGEPEA

SEQ ID NO: 189 - protein, 491 aa, synthetic construct
  REGION 1..491: note = PIV200976
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWLRRVNQKLDSIGSGEPEA

SEQ ID NO: 190 - protein, 491 aa, synthetic construct
  REGION 1..491: note = PIV200977
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDLEEVKEWLRRSNQKLDSIGSGEPEA

SEQ ID NO: 191 - protein, 491 aa, synthetic construct
  REGION 1..491: note = PIV200978
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDLEEVKEWIRRVNQKLDSIGSGEPEA

SEQ ID NO: 192 - protein, 491 aa, synthetic construct
  REGION 1..491: note = PIV200979
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDLEESKEWLRRVNQKLDSIGSGEPEA

SEQ ID NO: 193 - protein, 491 aa, synthetic construct
  REGION 1..491: note = PIV200980
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWLRRVNQKLDSIGSGEPEA

SEQ ID NO: 194 - protein, 491 aa, synthetic construct
  REGION 1..491: note = PIV200982
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPCIARLGCEACGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKVKSDFEEVKEWLRRVNQKLDSIGSGEPEA

SEQ ID NO: 195 - protein, 516 aa, synthetic construct
  REGION 1..516: note = PIV201082
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIEDKIEEILSKIYHIENEIARIKKLIGEAEPEA

SEQ ID NO: 196 - protein, 491 aa, synthetic construct
  REGION 1..491: note = PIV201105
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWIRRVNQKLDSIGSGEPEA

SEQ ID NO: 197 - protein, 491 aa, synthetic construct
  REGION 1..491: note = PIV201106
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWIRRVNQKLDSIGSGEPEA

SEQ ID NO: 198 - protein, 491 aa, synthetic construct
  REGION 1..491: note = PIV201107
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWIRRVNQKLDSIGSGEPEA

SEQ ID NO: 199 - protein, 491 aa, synthetic construct
  REGION 1..491: note = PIV201108
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWIRRVNQKLDSIGSGEPEA

SEQ ID NO: 200 - protein, 491 aa, synthetic construct
  REGION 1..491: note = PIV201109
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWIRRVNQKLDSIGSGEPEA

SEQ ID NO: 201 - protein, 491 aa, synthetic construct
  REGION 1..491: note = PIV201110
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGSGEPEA

SEQ ID NO: 202 - protein, 491 aa, synthetic construct
  REGION 1..491: note = PIV201111
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRVNQKLDSIGSGEPEA

SEQ ID NO: 203 - protein, 491 aa, synthetic construct
  REGION 1..491: note = PIV201112
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWIRRSNQKLDSIGSGEPEA

SEQ ID NO: 204 - protein, 491 aa, synthetic construct
  REGION 1..491: note = PIV201113
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIGSGEPEA

SEQ ID NO: 205 - protein, 491 aa, synthetic construct
  REGION 1..491: note = PIV201114
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWIRRVNQKLDSIGSGEPEA

SEQ ID NO: 206 - protein, 516 aa, synthetic construct
  REGION 1..516: note = PIV201115
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 207 - protein, 516 aa, synthetic construct
  REGION 1..516: note = PIV201116
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEESKEWIRRSNQKLDSIEDKIEEILSKIYHIENEIARIKKLIGEAEPEA

SEQ ID NO: 208 - protein, 491 aa, synthetic construct
  REGION 1..491: note = PIV201254
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGSGEPEA

SEQ ID NO: 209 - protein, 491 aa, synthetic construct
  REGION 1..491: note = PIV201255
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGSGEPEA

SEQ ID NO: 210 - protein, 491 aa, synthetic construct
  REGION 1..491: note = PIV201256
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGPPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGSGEPEA

SEQ ID NO: 211 - protein, 491 aa, synthetic construct
  REGION 1..491: note = PIV201257
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALNPIDISIELNKAKSDLEEVKEWIRRVNQKLDSIGSGEPEA

SEQ ID NO: 212 - protein, 491 aa, synthetic construct
  REGION 1..491: note = PIV210233
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGSGEPEA

SEQ ID NO: 213 - protein, 491 aa, synthetic construct
  REGION 1..491: note = PIV210234
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGSGEPEA

SEQ ID NO: 214 - protein, 491 aa, synthetic construct
  REGION 1..491: note = PIV210235
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGSGEPEA

SEQ ID NO: 215 - protein, 491 aa, synthetic construct
  REGION 1..491: note = PIV210236
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRVNQKLDSIGSGEPEA

SEQ ID NO: 216 - protein, 491 aa, synthetic construct
  REGION 1..491: note = PIV210237
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRSNQKLDSIGSGEPEA

SEQ ID NO: 217 - protein, 491 aa, synthetic construct
  REGION 1..491: note = PIV210238
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGSGEPEA

SEQ ID NO: 218 - protein, 491 aa, synthetic construct
  REGION 1..491: note = PIV210294
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGGSEPEA

SEQ ID NO: 219 - protein, 491 aa, synthetic construct
  REGION 1..491: note = PIV210295
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGGSEPEA

SEQ ID NO: 220 - protein, 491 aa, synthetic construct
  REGION 1..491: note = PIV210296
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGGSEPEA

SEQ ID NO: 221 - protein, 491 aa, synthetic construct
  REGION 1..491: note = PIV210297
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGGSEPEA

SEQ ID NO: 222 - protein, 491 aa, synthetic construct
  REGION 1..491: note = PIV210298
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGGSEPEA

SEQ ID NO: 223 - protein, 491 aa, synthetic construct
  REGION 1..491: note = PIV210299
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGGSEPEA

SEQ ID NO: 224 - protein, 491 aa, synthetic construct
  REGION 1..491: note = PIV210300
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGPLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGGSEPEA

SEQ ID NO: 225 - protein, 491 aa, synthetic construct
  REGION 1..491: note = PIV210301
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGGSEPEA

SEQ ID NO: 226 - protein, 491 aa, synthetic construct
  REGION 1..491: note = PIV210302
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAPSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGGSEPEA

SEQ ID NO: 227 - protein, 491 aa, synthetic construct
  REGION 1..491: note = PIV210303
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGGSEPEA

SEQ ID NO: 228 - protein, 491 aa, synthetic construct
  REGION 1..491: note = PIV210304
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGGSEPEA

SEQ ID NO: 229 - protein, 491 aa, synthetic construct
  REGION 1..491: note = PIV210305
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGGSEPEA

SEQ ID NO: 230 - protein, 491 aa, synthetic construct
  REGION 1..491: note = PIV210306
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGGSEPEA

SEQ ID NO: 231 - protein, 491 aa, synthetic construct
  REGION 1..491: note = PIV210307
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGGSEPEA

SEQ ID NO: 232 - protein, 491 aa, synthetic construct
  REGION 1..491: note = PIV210308
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGGSEPEA

SEQ ID NO: 233 - protein, 491 aa, synthetic construct
  REGION 1..491: note = PIV210309
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGGSEPEA

SEQ ID NO: 234 - protein, 491 aa, synthetic construct
  REGION 1..491: note = PIV210310
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSPGNLIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGGSEPEA

SEQ ID NO: 235 - protein, 491 aa, synthetic construct
  REGION 1..491: note = PIV210311
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLLLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRSNQKLDSIGGSEPEA

SEQ ID NO: 236 - protein, 491 aa, synthetic construct
  REGION 1..491: note = PIV210312
MPISILLIITTMIMASHCQIDITKLQHVGVLVNSPKGMKISQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLLLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIGGSEPEA

SEQ ID NO: 237 - protein, 485 aa, synthetic construct
  REGION 1..485: note = PIV211368
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIG

SEQ ID NO: 238 - protein, 485 aa, synthetic construct
  REGION 1..485: note = PIV211886
MPISILLIITTMIMASHCQIDITKLQHVGVLVNSPKGMKISQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIG

SEQ ID NO: 239 - protein, 485 aa, synthetic construct
  REGION 1..485: note = PIV211887
MPISILLIITTMIMASHCQIDITKLQHVGVLVNSPKGMKIPQNFETRYLILSLIPKIEDSNSCGDQQIKQYKRLLDRLIIPLYDGLRLQKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLQEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIG

SEQ ID NO: 240 - protein, 485 aa, synthetic construct
  REGION 1..485: note = PIV211888
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEESKEWIRRVNQKLDSIG

SEQ ID NO: 241 - protein, 485 aa, synthetic construct
  REGION 1..485: note = PIV211889
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRSNQKLDSIG

SEQ ID NO: 242 - protein, 485 aa, synthetic construct
  REGION 1..485: note = PIV211890
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTERFFGGVIGTIALGVATSAQITAAVALVEAKQARSDIEKLKEAIRDTNKAVQSVQSSVGNLIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIG

SEQ ID NO: 243 - protein, 485 aa, synthetic construct
MPISILLIITTMIMASHCQIDITKLQHVGVLVNSPKGMKIPQNFETRYLILSLIPKIEDSNSCGDQQIKQYKRLLDRLIIPLYDGLRLMKDVIVANQESNENTDPRTEQFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIG

SEQ ID NO: 244 - protein, 485 aa, synthetic construct
MPISILLIITTMIMASHCQIDITKLQHVGVLVNSPKGMKIPQNFETRYLILSLIPKIDDSNSCGDQQIKQYKRLLDRLIIPLYDGLRLMKDVIVANQESNENTDPRTEQFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIG

SEQ ID NO: 245 - protein, 485 aa, synthetic construct
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTEQFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIG

SEQ ID NO: 246 - protein, 485 aa, synthetic construct
MPISILLIITTMIMASHCQIDITKLQHVGVLVNSPKGMKIPQNFETRYLILSLIPKIDDSNSCGDQQIKQYKRLLDRLIIPLYDGLRLMKDVIVTNQESNENTDPRTEQFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPDDITLNNSVALDPIDISIELNKAKSDLEEVKEWIRRVNQKLDSIG

SEQ ID NO: 247 - protein, 485 aa, synthetic construct
MPISILLIITTMIMASHCQIDITKLQHVGVLVNSPKGMKIPQNFETRYLILSLIPKIEDSNSCGDQQIKQYKRLLDRLIIPLYDGLRLMKDVIVANQESNENTDPRTEQFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPADITLNNSVALDPIDISIELNKAKSDLEEVKEWIRRVNQKLDSIG

SEQ ID NO: 248 - protein, 485 aa, synthetic construct
MPISILLIITTMIMASHCQIDITKLQHVGVLVNSPKGMKIPQNFETRYLILSLIPKIDDSNSCGDQQIKQYKRLLDRLIIPLYDGLRLMKDVIVANQESNENTDPRTEQFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPADITLNNSVALDPIDISIELNKAKSDLEEVKEWIRRVNQKLDSIG

SEQ ID NO: 249 - protein, 485 aa, synthetic construct
MPISILLIITTMIMASHCQIDITKLQHVGVLVNSPKGMKIPQNFETRYLILSLIPKIEDSNSCGDQQIKQYKRLLDRLIIPLYDGLRLMKDVIVTNQESNENTDPRTEQFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPADITLNNSVALDPIDISIELNKAKSDLEEVKEWIRRVNQKLDSIG

SEQ ID NO: 250 - protein, 485 aa, synthetic construct
MPISILLIITTMIMASHCQIDITKLQHVGVLVNSPKGMKIPQNFETRYLILSLIPKIDDSNSCGDQQIKQYKRLLDRLIIPLYDGLRLMKDVIVTNQESNENTDPRTEQFFGGVIGTIALGVATSAQITAAVALVEAKQARSDIEKLKEAIRDTNKAVQSVQSSVGNPIVAIKSVQDYVNKEIVPSIARLGCEAAGLQLGIALTQHYSELTNIFGDNIGSLIEKGIKLQGIASLYRTNITEIFTTSTVDKYDIYDLLFTESIKVRVIDVDLNDYSITLQVRLPLLTRLLNTQIYKVDSISYNIQNREWYIPLPSHIMTKGAFLGGADVKECIEAFSSYICPSDPGFVLNHEMESCLSGNISQCPRTTVTSDIVPRYAFVNGGVVANCITTTCTCNGIGNRINQPPDQGVKIITHKECNTIGINGMLFNTNKEGTLAFYTPADITLNNSVALDPIDISIELNKAKSDLEEVKEWIRRVNQKLDSIG

(19) United States
(12) Patent Application Publication
(10) Pub. No.: US 2026/0232789 A1
(43) Pub. Date: Aug. 13, 2026

(54) STABILIZED PRE-FUSION PIV3 F PROTEINS

(71) Applicant: MSD International Business GmbH, Lucerne (CH)

(72) Inventors: Johannes Petrus Maria Langedijk, Amsterdam (NL); Mark Johannes Gerardus Bakkers, Haarsteeg (NL)

(73) Assignee: MSD International Business GmbH, Lucerne (CH)

(21) Appl. No.: 19/117,731

(22) PCT Filed: Oct. 5, 2023
(86) PCT No.: PCT/EP2023/077506

§ 371 Date: Apr. 2, 2025

(30) Foreign Application Priority Data
Oct. 6, 2022 (EP) 22200027.5

(51) Int. Cl. A61K 39/155; A61K 39/00; C07K 14/005; C12N 7/00; C12N 15/86

(52) U.S. Cl. CPC A61K 39/155; C07K 14/005; C12N 7/00; C12N 15/86; A61K 2039/53; C12N 2710/10043; C12N 2760/18622; C12N 2760/18634

(57) ABSTRACT

The present invention relates to stabilized pre-fusion human parainfluenza virus 3 (HPIV3) F protein, and fragments thereof. The invention also relates to nucleic acid molecules encoding such proteins and fragments, and to the use of the proteins, fragments and nucleic acid molecules.

### CROSS-REFERENCE TO RELATED APPLICATIONS

This application is a U.S. National Stage application of International Patent Application No. PCT/EP2023/077506, filed date Oct. 5, 2023, which claims priority to E.P. Application No. EP22200027.5, filed Oct. 6, 2022, each of which is hereby incorporated by reference in its entirety.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Oct. 19, 2022, is named CRU6092EPEPA1 sequence listing.xml and is 431,328 kilobytes in size.

### FIELD

The present invention relates to the field of medicine. The invention, in particular, relates to recombinant pre-fusion PIV3 F proteins, to nucleic acid molecules encoding the PIV3 F proteins, and uses thereof, e.g. in vaccines.

### BACKGROUND

Human parainfluenza type III (HPIV3) induces respiratory complications mainly in children and the immunocompromised; however, more recently, it was identified as a concern in the adult population as well. More than 11,000 hospitalizations of children per year in the US occur due to HPIV3 (Weinberg et., al., J Pediatr. 154:694-699, 2009) and HPIV3 is also an important cause of mortality, morbidity, and health care costs in other vulnerable populations (Ison et. al., Clin. Microbiol Rev 32, 2019). Most children of 5 years of age and older have antibodies against HPIV-3, indicating most children have experienced an HPIV3 infection by that age.

There is currently no vaccine and no specific antiviral treatment to prevent HPIV illness. Medical care is supportive, except for croup where the use of corticosteroids and nebulized epinephrine has been found to be beneficial.

Four serotypes of HPIV are known (HPIV-1 through-4), which are associated with distinct clinical presentations and seasonal incidence, with HPIV3 being the most prevalent and commonly presenting as bronchiolitis/pneumonia. Seasonal variations in the different serotypes and spontaneous outbreaks drive an overall variable incidence and complex epidemiology.

HPIV3 is an enveloped RNA virus in the Paramyxoviridae family of the order Mononegavirales. It has a genome of ~15,000 nucleotides in length that encodes six key proteins in the following gene sequence: 3′-N-P-M-F-HN-L-5. Virus-cell fusion results from coordinated action of the two envelope glycoproteins that comprise the viral entry machinery-a receptor binding protein, hemagglutinin neuraminidase (HN), and a fusion protein (F). Upon binding to sialic acid-containing target receptors, HN, a molecule with both receptor binding and cleaving activities, triggers and activates the F protein. The F protein fuses the viral and host-cell membranes by irreversible protein refolding from the labile pre-fusion conformation to the stable post-fusion conformation. Structures of both conformations have been determined for several paramyxoviruses, providing insight into the complex mechanism of this fusion protein. As type I membrane protein, the F protein is translated at the endoplasmic reticulum and transported through the Golgi apparatus and trans-Golgi network to the plasma membrane. Like other class I fusion proteins, the inactive precursor, PIV3 FO, requires cleavage into the disulfide-linked subunits F1 and F2 by appropriate host endoproteases, likely TMPRSS2, at a monobasic cleavage site. After this cleavage, F1 contains a hydrophobic fusion peptide (FP) at its N-terminus. In order to refold from the pre-fusion to the post-fusion conformation, the refolding region 1 (RR1) between residue 110 and 213, that includes the FP and heptad repeat A (HRA), (wherein the numbering is based on the numbering of amino acid residues in SEQ ID NO: 1) has to transform from an assembly of helices, loops and strands to a long continuous helix. The FP, located at the N-terminal segment of RR1, is then able to extend away from the viral membrane and to insert into the proximal membrane of the target cell. Next, the refolding region 2 (RR2), which forms the C-terminal stem in the pre-fusion F spike and includes the heptad repeat B (HRB), relocates to the other side of the PIV3 F head and binds the HRA coiled-coil trimer with the HRB domain to form the six-helix bundle. The formation of the RRI coiled-coil and relocation of RR2 to complete the six-helix bundle are the most dramatic structural changes that occur during the refolding process. Class I fusion proteins have been shown to be inherently unstable and structure-based stabilization of viral fusion protein in the prefusion conformation have been shown to induce superior neutralization and protection in animal models and clinical trials (Krarup et al., Nat Commun. 6:8143, 2015; De Taeye, Cell 163 (7): 1702-1715, 2015; Mclellan et al., Science. 342 (6158): 592-598, 2013; Stewart-Jones et al., PNAS 48:12265-12270, 2018; Crank et al., Science 365 (6452): 505-509, 2019, Sadoff et al., JID doi: 10.1093/infdis/jiab003 2021; Sadoff et al., NEJM, doi: 10.1056/NEJMoa2034201 2021), but until this date, still no vaccine is available and also no therapy exists for prevention or treatment of hPIV3.

Therefore, a need remains for efficient vaccines against PIV3, in particular vaccines comprising or based on PIV3 F proteins in the pre-fusion conformation. Indeed, vaccines, preferably indicated for pediatric and high-risk patients (e.g., elderly and COPD patients) could provide broad impact intervention far upstream of a serious illness thereby reducing HPIV3 overall incidence and associated morbidity and mortality. The present invention aims at providing means for obtaining such stable pre-fusion PIV3 F proteins for use in vaccinating against PIV3.

### SUMMARY

The present invention provides stable, recombinant, pre-fusion human parainfluenza type III (HPIV3) fusion (F) proteins, i.e. recombinant HPIV3 F proteins that are stabilized in the pre-fusion conformation, and fragments thereof. The pre-fusion HPIV3 F proteins, or fragments thereof, comprise at least one epitope that is specific to the pre-fusion conformation F protein, e.g. as determined by specific binding of an antibody that is specific for the pre-fusion conformation to the proteins. In certain preferred embodiments, the pre-fusion HPIV3 F proteins are soluble multimeric, preferably trimeric, proteins. The invention also provides nucleic acid molecules encoding the pre-fusion HPIV3 F proteins, or fragments thereof, as well as vectors, e.g. adenovectors, comprising such nucleic acid molecules.

The invention also relates to methods of stabilizing HPIV3 F proteins in the pre-fusion conformation, and to the pre-fusion PIV3 F proteins obtainable by said methods.

The invention further relates to compositions, preferably pharmaceutical compositions, comprising an PIV3 F protein, a nucleic acid molecule and/or a vector, as described herein, and to the use thereof in inducing an immune response against PIV3 F protein, in particular to the use thereof as a vaccine against PIV3. The invention also relates to methods for inducing an anti-parainfluenza virus type III (PIV3) immune response in a subject, comprising administering to the subject an effective amount of a pre-fusion HPIV3 F protein, a nucleic acid molecule encoding said HPIV3 F protein, and/or a vector comprising said nucleic acid molecule, as described herein. Preferably, the induced immune response is characterized by the induction of neutralizing antibodies to PIV3 and/or protective immunity against PIV3. In particular aspects, the invention relates to a method for inducing anti-parainfluenza virus type III (PIV3) F antibodies in a subject, comprising administering to the subject an effective amount of a pharmaceutical composition comprising a pre-fusion HPIV3 F protein, a nucleic acid molecule encoding said PIV3 F protein, and/or a vector comprising said nucleic acid molecule, as described herein.

### BRIEF DESCRIPTION OF THE DRAWINGS

FIG. 1: Schematic representation of the conserved elements of the PIV3 F protein in both the full-length, membrane bound protein (‘full-length’, top panel) and in the mature, soluble ectodomain (‘ectodomain’, bottom panel). The N-terminal F2 domain is preceded by a signal peptide sequence (SP) that is cleaved off during protein maturation. The fusion peptide (FP) is located at the N-terminus of F1. Heptad repeats A, B and C are indicated (HRA, HRB, HRC, respectively). Further indicated are the transmembrane region (TM) and cytoplasmic tail (CT). Soluble ectodomains may be equipped with a C-terminal GCN4 trimerization motif. Cleavage site between SP and F2 and between F2 and F1 are indicated with arrows.

FIG. 2: PIA174 binding to PIV3 preF in cell supernatant determined by biolayer interferometry. Quantitative octet measurements were performed by immobilizing antibody PIA174 to anti-human-IgG sensors and with PIV3 F in crude cell supernatant. Plotted is the initial binding rate. Cell culture medium of mock transfected cells (‘Mock’) and PIV3 F without stabilizing mutations and without GCN4 (‘Wildtype’, SEQ ID NO: 2) were taken along as negative controls. Measurements were performed at day of harvest (‘day 0’) and were repeated after 20-day storage at 4° C. (‘Day 20’). Single and double mutations were tested in a backbone with GCN4 trimerization domain and D452N mutation, as indicated.

FIG. 3: Analytical SEC profiles of different PIV3 F proteins with stabilizing mutations in crude cell supernatant. Indicated protein variants (black, solid lines), were compared to mock transfected supernatant (dashed lines). The peak between 4.4 and 4.5 minutes corresponds to the PIV3 preF trimer.

FIG. 4: PIA174 binding to PIV3 preF in cell supernatant determined by biolayer interferometry of single and all possible double combinations of stabilizing mutations. Quantitative octet measurements were performed by immobilizing antibody PIA174 to anti-human-IgG sensors and with PIV3 F in crude cell supernatant. Plotted is the initial binding rate. Cell culture medium of mock transfected cells (‘Mock’) and PIV3 F without stabilizing mutations and without GCN4 (‘Wildtype’, SEQ ID NO: 2) were taken along as controls. Measurements were performed at day of harvest. Single and double mutations were tested in a backbone with GCN4 trimerization domain and D452N mutation, as indicated.

FIG. 5: PIA174 binding to PIV3 preF in 5-fold diluted cell supernatant determined by biolayer interferometry of all possible combinations of selected stabilizing mutations. Quantitative octet measurements were performed by immobilizing antibody PIA174 to anti-human-IgG sensors and with PIV3 F in 5-fold diluted crude cell supernatant. Plotted is the initial binding rate. Combinations of mutations were tested in a backbone with GCN4 trimerization domain and D452N, Q89M, Q222I and L168P mutations, as indicated.

FIG. 6A-6C: Stabilized PIV3 preF in absence of GCN4 trimerization domain. FIG. 6A shows PIA174 binding to PIV3 preF PIV200941 (without GCN4 trimerization domain, but with D452N+Q89M+Q222I+L168P mutations) in cell supernatant as determined by biolayer interferometry. S470V and/or S477V were introduced in the stem of the PIV3 F protein. Plotted is the initial binding rate. FIG. 6B shows samples of FIG. 6A tested in analytical SEC. The peak at ~4.8 minutes corresponds to the PIV3 preF trimer. FIG. 6C shows PIA174 binding to unstabilized PIV3 preF without GCN4 trimerization domain (PIV190058, SEQ ID NO: 2), in cell supernatant as determined by biolayer interferometry. S470V or S477V ((PIV200960 and PIV200962, respectively) were introduced in the stem of the PIV3 F protein. Plotted is the initial binding rate.

FIG. 7A-7B: Further stabilization of PIV3 preF in absence of GCN4 trimerization domain. FIG. 7A shows PIA174 binding to a matrix of PIV3 preF designs without GCN4 trimerization domain in cell supernatant as determined by biolayer interferometry. Plotted is the initial binding rate. FIG. 7B shows samples of FIG. 7A tested in analytical SEC. The peak at ~4.8 minutes corresponds to the PIV3 preF trimer.

FIG. 8A-8B: Removal of S470V and S477V opens up the PIV3 preF trimer. FIG. 8A shows crude cell supernatants of PIV201105 and PIV201103 of FIG. 7B were tested in analytical SEC-MALS. Hydrodynamic radius and molecular weight (MW) of the main peaks (indicated with an arrow) were determined and are reported in FIG. 8B. The MALS signal corresponding to the molecular weight is shown as a dotted line for the respective peaks.

FIG. 9: Analytical SEC after heat stress. Indicated proteins in crude cell supernatant were incubated for 30 minutes at 4° C. (dashed line), 50° C. (black line) or 60° C. (gray line). Samples were then analyzed by analytical SEC to determine loss of PIV3 preF trimer. The backbone used contained S41P+Q89M+Q2221+N167P+L168P+D452N+S470V+S477V stabilizing mutations and did not have a heterologous trimerization domain.

FIG. 10: Stability of PIV3 preF variants. Crude cell supernatants of indicated proteins were analyzed by differential scanning fluorimetry (DSF) to determine the melting temperature.

FIG. 11A-11D: Characterization of purified PIV3 preF proteins. Proteins were purified from expiHEK supernatant using C-tag purification followed by size-exclusion chromatography. FIG. 11A shows an overview of the different protein designs, and their yield after purification.

FIG. 11B shows an SDS-PAGE under reducing and non-reducing conditions. Unprocessed PIV3 F proteins runs at ~50 kD. FIG. 11C shows SEC-MALS and FIG. 11D shows DSF analysis of purified proteins.

FIG. 12A shows a table of constructs used, indicating the absence (-) or presence of HR2 stem mutations S470V+S477V in the designs, and the absence (-) or introduction of various amino acid substitutions in the head domain of the PIV3 F protein. Single head mutations were assessed, except for the combination of Q889M+Q221I, the sidechains of which interact in the prefusion structure.

FIG. 12B shows prefusion PIV3 F trimer detection in the supernatant of cells transfected with variants indicated in FIG. 12A as determined by binding of prefusion-specific PIA174 antibody with biolayer interferometry (qOctet). Quantitative octet measurements were performed by immobilizing antibody PIA174 to anti-human-IgG sensors and with PIV3 F in crude cell supernatant. Plotted is the initial binding rate. FIG. C shows PIV3 F trimer detection in the supernatant of cells transfected with variants indicated in FIG. 12A as determined by analytical SEC. PIV3 F trimer (indicated with ‘T’) elutes between 4.6- and 4.8-minutes. Each panel compares the absence (dotted line) or presence (solid line) of S470V+S477V mutations in conjunction with amino acid substitution(s) in the head domain of PIV3 F (specific mutation indicated above each graph).

FIG. 13A shows a description of constructs used and corresponding melting temperature of PIV3 F trimer in supernatant of transfected Expi293 cells, as determined by differential scanning fluorimetry (DSF). The removal of single or double mutations from PIV211368 by reverting them to wildtype amino acids is indicated in bold.

FIG. 13B shows PIV3 F trimer yield in supernatant of cells transfected with variants indicated in FIG. 13A as determined by analytical SEC. PIV3 F trimer elutes between 4.6- and 4.8-minutes retention time.

FIG. 14A shows an analytical SEC of purified PIV3 F trimer PIV211368. Tagless PIV3 F was purified from cell-free supernatant of transfected Expi293 cells by ion exchange purification and polishing via size exclusion chromatography. FIG. 14B shows a table of purified PIV211368 PIV3 F protein characteristics, including yield, trimer size, hydrodynamic radius and melting temperature (DSF).

FIG. 14C shows slow-freeze stability of purified PIV3 F trimer PIV211368 in different buffers. The recovery of PIV3 F trimer after slowly freezing protein from 20° C. to −70° C. during a 24-hour period is compared to trimer recovery after 4° C. storage (histogram is average of n=5 individual measurements (open circles). Buffer composition: FB12; 20 mM Histidine, 75 mM NaCl, 5% sucrose, 0.02% PS80, 0.4% (w/w) EtOH, 0.1 mM EDTA, pH 6.5. PS4P4; 20 mM KHPO4, 75 mM NaCl, 4% Sucrose, PS20 0.01%, pH 6.5. TS5P2; 20 mM Tris, 75 mM NaCl, 5% sucrose, 0.02% PS20, 0.4% EtOH, pH7.5.

FIG. 15A shows an analytical SEC of purified PIV3 F trimer PIV210235. PIV3 F was purified from cell-free supernatant of transfected GnT1-cells by C-tag purification and polishing via size exclusion chromatography.

FIG. 15B shows a table of purified PIV210235 PIV3 F protein characteristics, including yield, trimer size, hydrodynamic radius and melting temperature (DSF).

FIG. 16: SDS-PAGE under reduced or non-reduced conditions, followed by Coomassie staining. The dotted circle indicates partial processing of PIV211368.

FIG. 17: Melting temperature of purified HPIV3 F trimer as determined by differential scanning fluorimetry (DSF). N=3 replicate measurements, and individual and average values are reported as grey and black solid lines, respectively.

FIG. 18: PIA174 binding to purified PIV3 preF proteins as determined by biolayer interferometry. The PIA174 antibody was immobilized to anti-human-IgG sensors. Plotted is the initial binding rate. A negative control (NC) protein that is not PIV3 preF, and the 1x kinetic buffer were taken along as negative controls.

### DETAILED DESCRIPTION

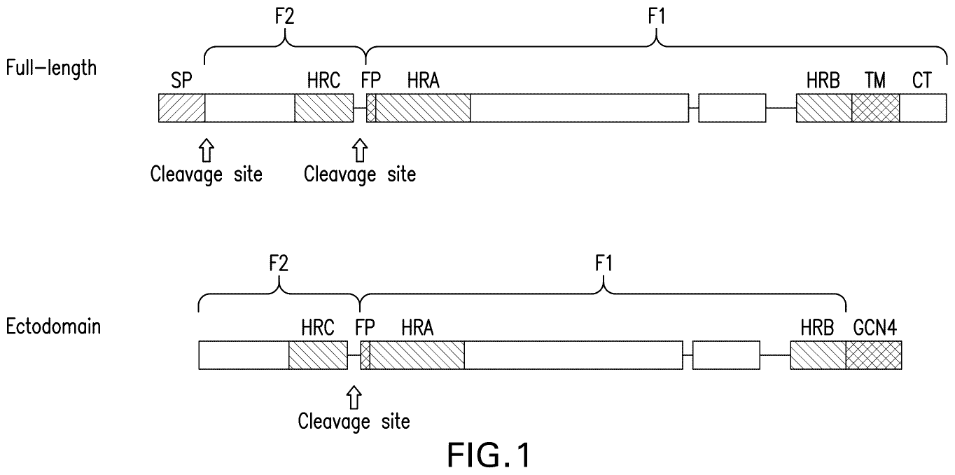

As described above, the fusion protein (F) of the parainfluenza virus (PIV3) is involved in fusion of the viral membrane with a host cell membrane, which is required for infection. PIV3 F mRNA is translated into a 539 amino acid precursor protein designated FO, which contains a signal peptide sequence at the N-terminus (e.g. amino acid residues 1-18 of SEQ ID NO: 1) which is removed by a signal peptidase in the endoplasmic reticulum. FO is cleaved, probably at the cell membrane, between amino acid residues 109 and 110 by cellular proteases (most likely TMPRSS2, or TMPRSS2-like enzymes) generating two domains or subunits designated F1 and F2. The F1 domain (amino acid residues 110-539) contains a hydrophobic fusion peptide at its N-terminus and the C-terminus contains the transmembrane (TM) (amino acid residues 494-516) and cytoplasmic region (amino acid residues 517-539). The F2 domain (amino acid residues 19-109) is covalently linked to F1 by one disulfide bridges (FIG. 1). The F1-F2 heterodimers are assembled as homotrimers on the virion surface. The mature ectodomain of the PIV3 F protein (comprising the amino acid residues 19-493) can be structurally divided in a globular head domain (amino acid residues 19-451), and a fibrous stem region (amino acid residues 452-484).

A vaccine against PIV3 infection is currently not yet available. One potential approach to producing a vaccine is a subunit vaccine based on purified PIV3 F protein. However, for this approach it is desirable that the purified PIV3 F protein is in a conformation which resembles the conformation of the pre-fusion state of PIV3 F protein, which is stable over time, i.e. remains in the pre-fusion conformation, e.g. as determined by specific binding of the PIV3 F protein to antibodies that are specific for the pre-fusion conformation to the PIV3 F protein, and can be produced in sufficient quantities. In addition, for a soluble, subunit-based vaccine, the PIV3 F protein needs to be truncated by deletion of the transmembrane (TM) and the cytoplasmic region to create a soluble secreted F protein ectodomain (sF). Because the TM region is responsible for membrane anchoring and increases stability, the ectodomain of the F protein is considerably more labile than the full-length protein and will even more readily refold into the post-fusion end-state. In order to obtain soluble F protein in the pre-fusion conformation that shows high expression levels and high stability, the pre-fusion conformation thus needs to be stabilized.

Because also the full length (membrane-bound) PIV3 F protein is metastable, the stabilization of the pre-fusion conformation is also desirable for the full length PIV3 F protein, i.e. including the TM and cytoplasmic region, e.g. for any live attenuated or vector based vaccine approaches.

Recently, a HPIV-3 protein variant was described, containing several stabilizing amino acid substitutions that stabilized the prefusion conformation (Stewart-Jones et al., PNAS 115 (48) 12265-12270, 2018). However, this variant has some limitations; i.e. i) Expression and stability of this PIV3 preF protein was insufficient for full development of a successful vaccine; ii) Several of these mutations were located at the surface of the protein, which may impact antigenicity and immunogenicity; and/or iii) This variant is C-terminally fused to the GCN4 trimerization domain which may impact immunogenicity and induce non-relevant antibodies to this trimerization domain that will not cross react with the virus and may hamper immunogenicity when this domain is used in other (future) vaccines which will increase its immunodominance.

Described herein are stabilized pre-fusion human parainfluenza virus 3 (HPIV3) F proteins, comprising an F1 and an F2 domain comprising an amino acid sequence of the F1 and F2 domain of an F protein of an HPIV3 strain, comprising a hydrophobic amino acid at position 470 and at position 477, wherein the numbering of the amino acid positions is according to the numbering is amino acid residues in SEQ ID NO: 1. Preferably, the proteins are trimeric. The hydrophobic amino acid at positions 470 and/or 477 can be any hydrophobic amino acid, including, but not limited to valine, leucine, isoleucine, methionine, and phenylalanine. The amino acid residues at position 470 and 477 may be the same hydrophobic amino acid, or different hydrophobic amino acids. In certain preferred embodiments, the hydrophobic amino acid at position 470 and/or 477 is valine (V), preferably both the amino acid at position 470 and 477 are valine (V).

The proteins may comprise one or more additional mutations. Thus, in certain embodiments, the amino acid residue at position 452 is N, and/or the amino acid residue at position 41 is P, and/or the amino acid residue at position 167 is P, and/or the amino acid residue at position 168 is P, and/or the amino acid sequence at position 335 is P, and/or the amino acid residue at position 89 is M and the amino acid residue at position 222 is I, and/or the amino acid residue at position 165 is P, and/or the amino acid residue at position 198 is L, and/or comprising a disulfide bridge between the amino acid residues 85 and 221, and/or between 186 and 195, wherein the numbering of the amino acid positions is according to the numbering is amino acid residues in SEQ ID NO: 1.

Furthermore, proteins are described wherein the amino acid residue at position 204 is D and/or the amino acid residue at position 367 is L and/or the amino acid residue at position 436 is P, and/or wherein the protein comprises a disulfide bridge between the amino acid residues 38 and 291.

The present invention provides proteins, comprising an F1 and an F2 domain comprising an amino acid sequence of the F1 and F2 domain of an F protein of an HPIV3 strain, wherein the amino acid residue at position 41 is P, and the amino acid residue at position 89 is M and the amino acid residue at position 222 is I, and the amino acid residue at position 168 is P, and the amino acid residue at position 470 is V and the amino acid residue at position 477 is V, and the amino acid residue at position 109 is Q, wherein the numbering of the amino acid positions is according to the numbering of amino acid residues in SEQ ID NO: 1.

The present invention provides stabilized trimeric pre-fusion HPIV-3 proteins that show high expression levels and increased stability. In addition, the proteins according to the invention are single chain proteins, i.e. the proteins are not processed (cleaved). Since the proteins are resistant to proteolysis, the manufacturability of the proteins is increased.

According to the invention it has been demonstrated that the presence of one or more of the specific amino acid residues at the indicated positions increases e.g. the stability of the HPIV3 F proteins and/or HPIV3 F protein ectodomains in the pre-fusion conformation, as compared to HPIV3 F protein without these amino acid residues at these positions. According to the invention, the specific amino acids can be either already present in the amino acid sequence or can be introduced by substitution (mutation) of the amino acid on that position into the specific amino acid according to the invention.

It is noted that the terms HPIV-3 and PIV-3 are used interchangeably throughout this application.

In certain embodiments, the proteins have an increased stability (thermostability) upon storage a 4° C., and/or at 50° C. and/or or 60° C., as compared to HPIV3 F proteins without the presence of these amino acid residues at these positions. With “stability upon storage”, it is meant that the proteins still display the at least one epitope specific for a pre-fusion specific antibody upon storage of the protein in solution (e.g. culture medium) at 4°, 50° C. and/or or 60° C. for a predetermined period of time.

In addition, or alternatively, the proteins may have an increased thermostability, e.g. as indicated by an increased melting temperature (measured by e.g. differential scanning fluorimetry).

The invention also provides fragments of the HPIV-3 F proteins. The term “fragment” as used herein refers to a HPIV3 polypeptide that has an amino-terminal (e.g. by cleaving off the signal sequence) and/or carboxy-terminal (e.g. by deleting the transmembrane region and/or cytoplasmic tail) and/or internal deletion, but wherein the remaining amino acid sequence is identical to the corresponding positions in the sequence of the HPIV3 F protein, for example, the full-length sequence of a HPIV3 F protein. It will be appreciated that for inducing an immune response and in general for vaccination purposes, a protein needs not to be full length nor have all its wild type functions, and fragments of the protein are equally useful. A fragment according to the invention is an immunologically active fragment, and typically comprises at least 15 amino acids, or at least 30 amino acids, of the HPIV3 F protein. In certain embodiments, a fragment comprises at least 50, 75, 100, 150, 200, 250, 300, 350, 400, 450, 460, 470, 480, 490, 500, or 510 amino acids of the HPIV3 F protein. In a preferred embodiment, the fragment is an HPIV3 F protein ectodomain, consisting of the amino acid residues 19-484 of the HPIV3 F protein.

In certain embodiments, the proteins or fragments thereof according to the invention do not comprise a signal sequence. It will be understood by the skilled person that signal sequences (sometimes referred to as signal peptide, targeting signal, localization signal, localization sequence, transit peptide, leader sequence or leader peptide) function to prompt a cell to translocate the protein, usually to the cellular membrane. Signal peptidase may cleave either during or after completion of translocation to generate a free signal peptide and a mature protein.

In certain embodiments, the PIV3 F protein ectodomain comprises a truncated F1 domain, preferably the truncated F1 domain does not comprise the transmembrane and cytoplasmic regions of the HPIV3 F protein. According to the invention said truncated F1 domain may comprise the amino acids 110-484, preferably the amino acids 110-485. In certain embodiments, the truncates F1 domain consists of the amino acids 110-484, preferably the amino acids 110-485 of the HPIV3 F protein.

In order to promote stable trimerization of the HPIV3 F ectodomains, a heterologous trimerization domain may be linked to the truncated F1 domain.

As described above, because the TM region is responsible for membrane anchoring and increases stability, the ectodomain of the F protein is considerably more labile than the full-length protein and will even more readily refold into the post-fusion end-state. In order to obtain stable soluble F protein in the pre-fusion conformation that shows high expression levels and high stability in certain embodiments a heterologous trimerization domain may be linked to the truncated F1 domain. The heterologous trimerization domain can be a GCN4 Leucine-Zipper domain. According to the invention, the heterologous trimerization domain preferably comprises, or consists of, the amino acid sequence of SEQ ID NO: 3. Alternative versions of GCN4 domains, or other heterologous trimerizations domains are also suitable according to the invention.

As used throughout the present application, the amino acid positions are given in reference to a wild type sequence of the HPIV3 F protein of SEQ ID NO: 1. As used in the present invention, the wording “the amino acid residue at position “x” of the F protein thus means the amino acid residue corresponding to the amino acid residue at position “x” in the HPIV3 F protein of SEQ ID NO: 1. Note that, in the numbering system used throughout this application 1 refers to the N-terminal amino acid of an immature FO protein (SEQ ID NO: 1). When an F protein of another HPIV-3 strain is used, the amino acid positions of the F protein are to be numbered with reference to the numbering of the F protein of SEQ ID NO: 1 by aligning the sequences of the other HPIV3 F protein with the F protein of SEQ ID NO: 1 with the insertion of gaps as needed. Sequence alignments can be done using methods well known in the art, e.g. by CLUSTALW, Bioedit or CLC Workbench.

The stabilized pre-fusion human parainfluenza virus 3 (HPIV3) F protein ectodomains, comprise a truncated F1 domain and an F2 domain comprising an amino acid sequence of the F1 and F2 domain of an F protein of an HPIV3 strain, wherein the amino acid residue at position 470 and/or 477 is a hydrophobic amino acid, wherein the protein does not comprise a heterologous trimerization domain, and wherein the numbering of the amino acid positions is according to the numbering is amino acid residues in SEQ ID NO: 1.

According to the present invention, it has been demonstrated that stable soluble trimeric pre-fusion PIV-3 ectodomains (i.e. soluble trimeric pre-fusion PIV-3 proteins) can be obtained without the presence of a heterologous trimerization domain, when the amino acid residue at position 470 and/or the amino acid residue at position 477 is a hydrophobic amino acid, preferably when the amino acid residues at both position 470 and 477 are hydrophobic.

The hydrophobic amino acid at positions 470 and/or 477 can be any hydrophobic amino acid, including, but not limited to, valine, leucine, isoleucine, methionine, and phenylalanine. The amino acid residues at position 470 and 477 may be the same hydrophobic amino acid, or different hydrophobic amino acids. In certain preferred embodiments, the hydrophobic amino acid at position 470 and/or 477 is valine (V), preferably both the amino acid at position 470 and 477 are valine (V).

In certain embodiments, the truncated F1 domain does not comprise the transmembrane and cytoplasmic regions. Preferably, the truncated F1 domain comprises the amino acids 110-484, preferably 110-485. In certain embodiment, the truncated F1 domain consists of the amino acids 110-484, preferably the amino acids 110-485 of the HPIV3 F protein.

In certain embodiments, furthermore the amino acid residue at position 95 is A, and/or the amino acid residue at position 441 is A, and/or the amino acid residue at position 58 is D.

In certain embodiments, the protein comprises an amino acid selected from the group consisting of SEQ ID NO: 243-250, or a fragment thereof. Preferably, the protein comprises the amino acid sequence of SEQ ID NO: 243.

In certain embodiments, the proteins do not comprise a signal sequence (i.e. corresponding to amino acids 1-18 of SEQ ID NO: 1).

In certain embodiments, the proteins do not comprise a C-terminal tag (C-tag).

As used throughout the present application nucleotide sequences are provided from 5′ to 3′ direction, and amino acid sequences from N-terminus to C-terminus, as custom in the art.

An amino acid according to the invention can be any of the twenty naturally occurring (or ‘standard’ amino acids). The standard amino acids can be divided into several groups based on their properties. Important factors are charge, hydrophilicity or hydrophobicity, size and functional groups. These properties are important for protein structure and protein-protein interactions. Some amino acids have special properties such as cysteine, that can form covalent disulfide bonds (or disulfide bridges) to other cysteine residues, proline that induces turns of the protein backbone, and glycine that is more flexible than other amino acids. Table 1 shows the abbreviations and properties of the standard amino acids.

It will be appreciated by a skilled person that the mutations can be made to the protein by routine molecular biology procedures. The mutations according to the invention preferably result in increased expression levels and/or increased stabilization of the pre-fusion PIV3 F proteins as compared to PIV3 F proteins that do not comprise these mutation(s).

The present invention further provides nucleic acid molecules encoding the PIV3 F proteins according to the invention. The nucleic acid molecule may be DNA or RNA. According to the invention, the RNA may be mRNA, modified mRNA, self-replicating RNA, or circular mRNA.

In preferred embodiments, the nucleic acid molecules encoding the proteins according to the invention are codon-optimized for expression in mammalian cells, preferably human cells. Methods of codon-optimization are known and have been described previously (e.g. WO 96/09378). A sequence is considered codon-optimized if at least one non-preferred codon as compared to a wild type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in http://www.kazusa.or.jp/codon. Preferably more than one non-preferred codon, preferably most or all non-preferred codons, are replaced by codons that are more preferred. Preferably the most frequently used codons in an organism are used in a codon-optimized sequence. Replacement by preferred codons generally leads to higher expression.

It will be understood by a skilled person that numerous different polynucleotides and nucleic acid molecules can encode the same protein as a result of the degeneracy of the genetic code. It is also understood that skilled persons may, using routine techniques, make nucleotide substitutions that do not affect the protein sequence encoded by the nucleic acid molecules to reflect the codon usage of any particular host organism in which the proteins are to be expressed. Therefore, unless otherwise specified, a “nucleotide sequence encoding an amino acid sequence” includes all nucleotide sequences that are degenerate versions of each other and that encode the same amino acid sequence. Nucleotide sequences that encode proteins and RNA may or may not include introns.

Nucleic acid sequences can be cloned using routine molecular biology techniques, or generated de novo by DNA synthesis, which can be performed using routine procedures by service companies having business in the field of DNA synthesis and/or molecular cloning (e.g. GeneArt, GenScripts, Invitrogen, Eurofins).

The invention also provides vectors comprising a nucleic acid molecule as described above. In certain embodiments, a nucleic acid molecule according to the invention thus is part of a vector.

In certain embodiments of the invention, the vector is an adenovirus vector. An adenovirus according to the invention belongs to the family of the Adenoviridae, and preferably is one that belongs to the genus Mastadenovirus. It can be a human adenovirus, but also an adenovirus that infects other species, including but not limited to a bovine adenovirus (e.g., bovine adenovirus 3, BAdV3), a canine adenovirus (e.g., CAdV2), a porcine adenovirus (e.g., PAdV3 or 5), or a simian adenovirus (which includes a monkey adenovirus and an ape adenovirus, such as a chimpanzee adenovirus or a gorilla adenovirus). Preferably, the adenovirus is a human adenovirus (HAdV, or AdHu), or a simian adenovirus such as chimpanzee or gorilla adenovirus (ChAd, AdCh, or SAdV), or a rhesus monkey adenovirus (RhAd). In the invention, a human adenovirus is meant if referred to as Ad without indication of species, e.g., the brief notation “Ad26” means the same as HAdV26, which is human adenovirus serotype 26. Also as used herein, the notation “rAd” means recombinant adenovirus, e.g., “rAd26” refers to recombinant human adenovirus 26.

Most advanced studies have been performed using human adenoviruses, and human adenoviruses are preferred according to certain aspects of the invention. In certain preferred embodiments, a recombinant adenovirus according to the invention is based upon a human adenovirus. In preferred embodiments, the recombinant adenovirus is based upon a human adenovirus serotype 5, 11, 26, 34, 35, 48, 49, 50, 52, etc. According to a particularly preferred embodiment of the invention, an adenovirus is a human adenovirus of serotype 26. Advantages of these serotypes include a low seroprevalence and/or low pre-existing neutralizing antibody titers in the human population, and experience with use in human subjects in clinical trials.

Simian adenoviruses generally also have a low seroprevalence and/or low pre-existing neutralizing antibody titers in the human population, and a significant amount of work has been reported using chimpanzee adenovirus vectors (e.g., U.S. Pat. No. 6,083,716; WO 2005/071093; WO 2010/086189; WO 2010/085984; Farina et al, 2001, J Virol 75:11603-13; Cohen et al, 2002, J Gen Virol 83:151-55; Kobinger et al, 2006, Virology 346:394-401; Tatsis et al., 2007, Molecular Therapy 15:608-17; see also review by Bangari and Mittal, 2006, Vaccine 24:849-62; and review by Lasaro and Ertl, 2009, Mol Ther 17:1333-39). Hence, in other embodiments, the recombinant adenovirus according to the invention is based upon a simian adenovirus, e.g. a chimpanzee adenovirus. In certain embodiments, the recombinant adenovirus is based upon simian adenovirus type 1, 7, 8, 21, 22, 23, 24, 25, 26, 27.1, 28.1, 29, 30, 31.1, 32, 33, 34, 35.1, 36, 37.2, 39, 40.1, 41.1, 42.1, 43, 44, 45, 46, 48, 49, 50 or SA7P. In certain embodiments, the recombinant adenovirus is based upon a chimpanzee adenovirus such as ChAdOx 1 (see, e.g., WO 2012/172277), or ChAdOx 2 (see, e.g., WO 2018/215766). In certain embodiments, the recombinant adenovirus is based upon a chimpanzee adenovirus such as BZ28 (see, e.g., WO 2019/086466). In certain embodiments, the recombinant adenovirus is based upon a gorilla adenovirus such as BLY6 (see, e.g., WO 2019/086456), or BZI (see, e.g., WO 2019/086466).

In a preferred embodiment of the invention, the adenoviral vectors comprise capsid proteins from rare serotypes, e.g. including Ad26. In the typical embodiment, the vector is an rAd26 virus. An “adenovirus capsid protein” refers to a protein on the capsid of an adenovirus (e.g., Ad26, Ad35, rAd48, rAd5HVR48 vectors) that is involved in determining the serotype and/or tropism of a particular adenovirus. Adenoviral capsid proteins typically include the fiber, penton and/or hexon proteins. As used herein a “capsid protein” for a particular adenovirus, such as an “Ad26 capsid protein” can be, for example, a chimeric capsid protein that includes at least a part of an Ad26 capsid protein. In certain embodiments, the capsid protein is an entire capsid protein of Ad26. In certain embodiments, the hexon, penton, and fiber are of Ad26.

One of ordinary skill in the art will recognize that elements derived from multiple serotypes can be combined in a single recombinant adenovirus vector. Thus, a chimeric adenovirus that combines desirable properties from different serotypes can be produced. Thus, in some embodiments, a chimeric adenovirus of the invention could combine the absence of pre-existing immunity of a first serotype with characteristics such as temperature stability, assembly, anchoring, production yield, redirected or improved infection, stability of the DNA in the target cell, and the like. See for example WO 2006/040330 for chimeric adenovirus Ad5HVR48, that includes an Ad5 backbone having partial capsids from Ad48, and also e.g. WO 2019/086461 for chimeric adenoviruses Ad26HVRPtr1, Ad26HVRPtr12, and Ad26HVRPtr13, that include an Ad26 virus backbone having partial capsid proteins of Ptr1, Ptr12, and Ptr13, respectively)

In certain preferred embodiments the recombinant adenovirus vector useful in the invention is derived mainly or entirely from Ad26 (i.e., the vector is rAd26). In some embodiments, the adenovirus is replication deficient, e.g., because it contains a deletion in the E1 region of the genome. For adenoviruses being derived from non-group C adenovirus, such as Ad26 or Ad35, it is typical to exchange the E4-orf6 coding sequence of the adenovirus with the E4-orf6 of an adenovirus of human subgroup C such as Ad5. This allows propagation of such adenoviruses in well-known complementing cell lines that express the E1 genes of Ad5, such as for example 293 cells, PER.C6 cells, and the like (see, e.g., Havenga, et al., 2006, J Gen Virol 87:2135-43; WO 03/104467). However, such adenoviruses will not be capable of replicating in non-complementing cells that do not express the E1 genes of Ad5.

The preparation of recombinant adenoviral vectors is well known in the art. Preparation of rAd26 vectors is described, for example, in WO 2007/104792 and in Abbink et al., (2007) Virol 81 (9): 4654-63. Exemplary genome sequences of Ad26 are found in GenBank Accession EF 153474 and in SEQ ID NO: 1 of WO 2007/104792. Examples of vectors useful for the invention for instance include those described in WO2012/082918, the disclosure of which is incorporated herein by reference in its entirety.

Typically, a vector useful in the invention is produced using a nucleic acid comprising the entire recombinant adenoviral genome (e.g., a plasmid, cosmid, or baculovirus vector). Thus, the invention also provides isolated nucleic acid molecules that encode the adenoviral vectors of the invention. The nucleic acid molecules of the invention can be in the form of RNA or in the form of DNA obtained by cloning or produced synthetically. The DNA can be double-stranded or single-stranded.

The adenovirus vectors useful in the invention are typically replication deficient. In these embodiments, the virus is rendered replication deficient by deletion or inactivation of regions critical to replication of the virus, such as the E1 region. The regions can be substantially deleted or inactivated by, for example, inserting a gene of interest, such as a gene encoding the stabilized pre-fusion PIV3 F protein (usually linked to a promoter), or a gene encoding the pre-fusion PIV3 F protein fragment (usually linked to a promoter) within the region. In some embodiments, the vectors of the invention can contain deletions in other regions, such as the E2, E3 or E4 regions, or insertions of heterologous genes linked to a promoter within one or more of these regions. For E2- and/or E4-mutated adenoviruses, generally E2- and/or E4-complementing cell lines are used to generate recombinant adenoviruses. Mutations in the E3 region of the adenovirus need not be complemented by the cell line, since E3 is not required for replication.

A packaging cell line is typically used to produce sufficient amounts of adenovirus vectors for use in the invention. A packaging cell is a cell that comprises those genes that have been deleted or inactivated in a replication deficient vector, thus allowing the virus to replicate in the cell. Suitable packaging cell lines for adenoviruses with a deletion in the E1 region include, for example, PER. C6, 911, 293, and E1 A549.

In a preferred embodiment of the invention, the vector is an adenovirus vector, and more preferably a rAd26 vector, most preferably a rAd26 vector with at least a deletion in the E1 region of the adenoviral genome, e.g. such as that described in Abbink, J Virol, 2007. 81 (9): p. 4654-63, which is incorporated herein by reference. Typically, the nucleic acid sequence encoding the pre-fusion PIV3 F protein is cloned into the E1 and/or the E3 region of the adenoviral genome.

Host cells comprising the nucleic acid molecules encoding the pre-fusion PIV3 F proteins form also part of the invention. The pre-fusion PIV3 F proteins may be produced through recombinant DNA technology involving expression of the molecules in host cells, e.g. Chinese hamster ovary (CHO) cells, tumor cell lines, BHK cells, human cell lines such as HEK293 cells, PER.C6 cells, or yeast, fungi, insect cells, and the like, or transgenic animals or plants. In certain embodiments, the cells are from a multicellular organism, in certain embodiments they are of vertebrate or invertebrate origin. In certain embodiments, the cells are mammalian cells. In certain embodiments, the cells are human cells. In general, the production of a recombinant proteins, such the pre-fusion PIV3 F proteins of the invention, in a host cell comprises the introduction of a heterologous nucleic acid molecule encoding the protein in expressible format into the host cell, culturing the cells under conditions conducive to expression of the nucleic acid molecule and allowing expression of the protein in said cell. The nucleic acid molecule encoding a protein in expressible format may be in the form of an expression cassette, and usually requires sequences capable of bringing about expression of the nucleic acid, such as enhancer(s), promoter, polyadenylation signal, and the like. The person skilled in the art is aware that various promoters can be used to obtain expression of a gene in host cells. Promoters can be constitutive or regulated, and can be obtained from various sources, including viruses, prokaryotic, or eukaryotic sources, or artificially designed.

Cell culture media are available from various vendors, and a suitable medium can be routinely chosen for a host cell to express the protein of interest, here the pre-fusion PIV3 F proteins. The suitable medium may or may not contain serum.

A “heterologous nucleic acid molecule” (also referred to herein as ‘transgene’) is a nucleic acid molecule that is not naturally present in the host cell. It is introduced into for instance a vector by standard molecular biology techniques. A transgene is generally operably linked to expression control sequences. This can for instance be done by placing the nucleic acid encoding the transgene(s) under the control of a promoter. Further regulatory sequences may be added. Many promoters can be used for expression of a transgene(s), and are known to the skilled person, e.g. these may comprise viral, mammalian, synthetic promoters, and the like. A non-limiting example of a suitable promoter for obtaining expression in eukaryotic cells is a CMV-promoter (U.S. Pat. No. 5,385,839), e.g. the CMV immediate early promoter, for instance comprising nt.-735 to +95 from the CMV immediate early gene enhancer/promoter. A polyadenylation signal, for example the bovine growth hormone poly A signal (U.S. Pat. No. 5,122,458), may be present behind the transgene(s). Alternatively, several widely used expression vectors are available in the art and from commercial sources, e.g. the pcDNA and pEF vector series of Invitrogen, pMSCV and pTK-Hyg from BD Sciences, pCMV-Script from Stratagene, etc, which can be used to recombinantly express the protein of interest, or to obtain suitable promoters and/or transcription terminator sequences, poly A sequences, and the like.

The cell culture can be any type of cell culture, including adherent cell culture, e.g. cells attached to the surface of a culture vessel or to microcarriers, as well as suspension culture. Most large-scale suspension cultures are operated as batch or fed-batch processes because they are the most straightforward to operate and scale up. Nowadays, continuous processes based on perfusion principles are becoming more common and are also suitable. Suitable culture media are also well known to the skilled person and can generally be obtained from commercial sources in large quantities, or custom-made according to standard protocols. Culturing can be done for instance in dishes, roller bottles or in bioreactors, using batch, fed-batch, continuous systems and the like. Suitable conditions for culturing cells are known (see e.g. Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9)).

The invention further provides compositions comprising a pre-fusion PIV3 F protein, and/or fragment thereof, and/or a nucleic acid molecule, and/or a vector, as described herein. The invention thus provides compositions comprising a pre-fusion PIV3 F protein, or fragment thereof, that displays an epitope that is present in a pre-fusion conformation of the PIV3 F protein but is absent in the post-fusion conformation. The invention also provides compositions comprising a nucleic acid molecule and/or a vector, encoding such pre-fusion PIV3 F protein or fragment. The invention further provides pharmaceutical compositions, e.g. vaccine compositions, comprising a pre-fusion PIV3 F protein, a PIV3 F protein fragment, and/or a nucleic acid molecule, and/or a vector, as described above and one or more pharmaceutically acceptable excipients.

The invention also provides the use of a stabilized pre-fusion PIV3 F protein (fragment), a nucleic acid molecule, and/or a vector, according to the invention, for inducing an immune response against PIV3 F protein in a subject. Further provided are methods for inducing an immune response against PIV3 F protein in a subject, comprising administering to the subject a pre-fusion PIV3 F protein (fragment), and/or a nucleic acid molecule, and/or a vector, according to the invention. Also provided are pre-fusion PIV3 F protein (fragments), nucleic acid molecules, and/or vectors, according to the invention for use in inducing an immune response against PIV3 F protein in a subject. Further provided is the use of the pre-fusion PIV3 F protein (fragments), and/or nucleic acid molecules, and/or vectors according to the invention for the manufacture of a medicament for use in inducing an immune response against PIV3 F protein in a subject. The invention in particular provides pre-fusion PIV3 F protein (fragments), and/or nucleic acid molecules, and/or vectors according to the invention for use as a vaccine.

The pre-fusion PIV3 F protein (fragments), nucleic acid molecules, or vectors of the invention may be used for prevention (prophylaxis) and/or treatment of PIV3 infections. In certain embodiments, the prevention and/or treatment may be targeted at patient groups that are susceptible PIV3 infection. Such patient groups include, but are not limited to e.g., the elderly (e.g. >50 years old, ≥60 years old, and preferably >65 years old), the young (e.g. ≤5 years old, ≤1 year old), pregnant women (for maternal immunization), and hospitalized patients and patients who have been treated with an antiviral compound but have shown an inadequate antiviral response.

The pre-fusion PIV3 F proteins, fragments, nucleic acid molecules and/or vectors according to the invention may be used in stand-alone treatment and/or prophylaxis of a disease or condition caused by PIV3, or in combination with other prophylactic and/or therapeutic treatments, such as (existing or future) vaccines, antiviral agents and/or monoclonal antibodies.

The invention further provides methods for preventing and/or treating PIV3 infection in a subject utilizing the pre-fusion PIV3 F proteins or fragments thereof, nucleic acid molecules and/or vectors according to the invention. In a specific embodiment, a method for preventing and/or treating PIV3 infection in a subject comprises administering to a subject in need thereof an effective amount of a pre-fusion PIV3 F protein (fragment), nucleic acid molecule and/or a vector, as described above. A therapeutically effective amount refers to an amount of a protein, nucleic acid molecule or vector, that is effective for preventing, ameliorating and/or treating a disease or condition resulting from infection by PIV3. Prevention encompasses inhibiting or reducing the spread of PIV3 or inhibiting or reducing the onset, development or progression of one or more of the symptoms associated with infection by PIV3. Amelioration as used in herein may refer to the reduction of visible or perceptible disease symptoms, viremia, or any other measurable manifestation of PIV3 infection.

For administering to subjects, such as humans, the invention may employ pharmaceutical compositions comprising a pre-fusion PIV3 F protein (fragment), a nucleic acid molecule and/or a vector as described herein, and a pharmaceutically acceptable carrier or excipient. In the present context, the term “pharmaceutically acceptable” means that the carrier or excipient, at the dosages and concentrations employed, will not cause any unwanted or harmful effects in the subjects to which they are administered. Such pharmaceutically acceptable carriers and excipients are well known in the art (see Remington's Pharmaceutical Sciences, 18th edition,

A. R. Gennaro, Ed., Mack Publishing Company [1990]; Pharmaceutical Formulation Development of Peptides and Proteins, S. Frokjaer and L. Hovgaard, Eds., Taylor & Francis

; and Handbook of Pharmaceutical Excipients, 3rd edition, A. Kibbe, Ed., Pharmaceutical

Press [2000]). The PIV3 F proteins, or nucleic acid molecules, preferably are formulated and administered as a sterile solution although it may also be possible to utilize lyophilized preparations. Sterile solutions are prepared by sterile filtration or by other methods known per se in the art. The solutions are then lyophilized or filled into pharmaceutical dosage containers. The pH of the solution generally is in the range of pH 3.0 to 9.5, e.g. pH 5.0 to 7.5. The PIV3 F proteins typically are in a solution having a suitable pharmaceutically acceptable buffer, and the composition may also contain a salt. Optionally stabilizing agent may be present, such as albumin. In certain embodiments, detergent is added. In certain embodiments, the PIV3 F proteins may be formulated into an injectable preparation.

In certain embodiments, a composition according to the invention further comprises one or more adjuvants. Adjuvants are known in the art to further increase the immune response to an applied antigenic determinant. The terms “adjuvant” and “immune stimulant” are used interchangeably herein and are defined as one or more substances that cause stimulation of the immune system. In this context, an adjuvant is used to enhance an immune response to the PIV3 F proteins of the invention. Examples of suitable adjuvants include aluminium salts such as aluminium hydroxide and/or aluminium phosphate; oil-emulsion compositions (or oil-in-water compositions), including squalene-water emulsions, such as MF59 (see e.g. WO 90/14837); saponin formulations, such as for example QS21 and Immunostimulating Complexes (ISCOMS) (see e.g. U.S. Pat. No. 5,057,540; WO 90/03184, WO 96/11711, WO 2004/004762, WO 2005/002620); bacterial or microbial derivatives, examples of which are monophosphoryl lipid A (MPL), 3-O-deacylated MPL (3dMPL), CpG-motif containing oligonucleotides, ADP-ribosylating bacterial toxins or mutants thereof, such as E. coli heat labile enterotoxin LT, cholera toxin CT, and the like; eukaryotic proteins (e.g. antibodies or fragments thereof (e.g. directed against the antigen itself or CD1a, CD3, CD7, CD80) and ligands to receptors (e.g. CD40L, GMCSF, GCSF, etc), which stimulate immune response upon interaction with recipient cells. In certain embodiments the compositions of the invention comprise aluminium as an adjuvant, e.g. in the form of aluminium hydroxide, aluminium phosphate, aluminium potassium phosphate, or combinations thereof, in concentrations of 0.05-5 mg, e.g. from 0.075-1.0 mg, of aluminium content per dose.

In other embodiments, the compositions do not comprise adjuvants.

In certain embodiments, the invention provides methods for making a vaccine against respiratory syncytial virus (PIV3), comprising providing an PIV3 F protein (fragment), nucleic acid or vector according to the invention and formulating it into a pharmaceutically acceptable composition. The term “vaccine” refers to an agent or composition containing an active component effective to induce a certain degree of immunity in a subject against a certain pathogen or disease, which will result in at least a decrease (up to complete absence) of the severity, duration or other manifestation of symptoms associated with infection by the pathogen or the disease. In the present invention, the vaccine comprises an effective amount of a pre-fusion PIV3 F protein (fragment) and/or a nucleic acid molecule encoding a pre-fusion PIV3 F protein, and/or a vector comprising said nucleic acid molecule, which results in an effective immune response against PIV3. This provides a method of preventing serious lower respiratory tract disease leading to hospitalization and the decrease in frequency of complications such as pneumonia and bronchiolitis due to PIV3 infection and replication in a subject. The term “vaccine” according to the invention implies that it is a pharmaceutical composition, and thus typically includes a pharmaceutically acceptable diluent, carrier or excipient. It may or may not comprise further active ingredients. In certain embodiments it may be a combination vaccine that further comprises other components that induce an immune response, e.g. against other proteins of PIV3 and/or against other infectious agents, e.g. against RSV, HMPV and/or influenza. The administration of further active components may for instance be done by separate administration or by administering combination products of the vaccines of the invention and the further active components.

Administration of the compositions according to the invention can be performed using standard routes of administration. Non-limiting embodiments include parenteral administration, such as intradermal, intramuscular, subcutaneous, transcutaneous, or mucosal administration, e.g. intranasal, oral, and the like. In one embodiment a composition is administered by intramuscular injection. The skilled person knows the various possibilities to administer a composition, e.g. a vaccine in order to induce an immune response to the antigen(s) in the vaccine.

A subject as used herein preferably is a mammal, for instance a rodent, e.g. a mouse, a cotton rat, or a non-human-primate, or a human. Preferably, the subject is a human subject.

The proteins, fragments, nucleic acid molecules, vectors, and/or compositions may also be administered, either as prime, or as boost, in a homologous or heterologous prime-boost regimen. If a boosting vaccination is performed, typically, such a boosting vaccination will be administered to the same subject at a time between one week and one year, preferably between two weeks and four months, after administering the composition to the subject for the first time (which is in such cases referred to as ‘priming vaccination’). In certain embodiments, the administration comprises a prime and at least one booster administration.

The invention further provides methods for making a vaccine against PIV3, comprising providing a recombinant human adenovirus of serotype 26 that comprises nucleic acid encoding a pre-fusion PIV3 F protein or fragment thereof as described herein, propagating said recombinant adenovirus in a culture of host cells, isolating and purifying the recombinant adenovirus, and bringing the recombinant adenovirus in a pharmaceutically acceptable composition. In certain embodiments, provided herein are methods of producing an adenoviral particle comprising a nucleic acid molecule encoding a PIV3 F protein or fragment thereof (transgene). The methods comprise (a) contacting a host cell of the invention with an adenoviral vector of the invention and (b) growing the host cell under conditions wherein the adenoviral particle comprising the transgene is produced. Recombinant adenovirus can be prepared and propagated in host cells, according to well-known methods, which entail cell culture of the host cells that are infected with the adenovirus. The cell culture can be any type of cell culture, including adherent cell culture, e.g. cells attached to the surface of a culture vessel or to microcarriers, as well as suspension culture.

Most large-scale suspension cultures are operated as batch or fed-batch processes because they are the most straightforward to operate and scale up. Nowadays, continuous processes based on perfusion principles are becoming more common and are also suitable (see, e.g., WO 2010/060719, and WO 2011/098592, both incorporated by reference herein, which describe suitable methods for obtaining and purifying large amounts of recombinant adenoviruses).

The invention further provides an isolated recombinant nucleic acid that forms the genome of a recombinant human adenovirus of serotype 26 that comprises nucleic acid encoding a PIV3 F protein or fragment thereof, as described herein.

In addition, the proteins of the invention may be used as diagnostic tool, for example to test the immune status of an individual by establishing whether there are antibodies in the serum of such individual capable of binding to the protein of the invention. The invention thus also relates to an in vitro diagnostic method for detecting the presence of an PIV3 infection in a patient said method comprising the steps of a) contacting a biological sample obtained from said patient with a protein according to the invention; and b) detecting the presence of antibody-protein complexes.

The invention is further illustrated in the following examples. The examples do not limit the invention in any way. They merely serve to clarify the invention.

### EXAMPLES

### Example 1: Instability of Soluble PIV3 F Ectodomain Protein

A plasmid encoding the wildtype PIV3 F protein ectodomain in which the transmembrane and cytoplasmic tail were replaced with a C-tag (SEQ ID NO: 2) was synthesized and codon-optimized at Genscript. The construct was cloned into pCDNA2004 by standard methods widely known within the field involving site-directed mutagenesis and PCR and sequenced. The protein was expressed in the expi293F cell system. Expi293F cells were transiently transfected using ExpiFectamine (Life Technologies) according to the manufacturer's instructions and cultured for 3 days at 37° C. and 10% CO2. The culture supernatant was collected, and cells and cellular debris were removed by centrifugation for 5 minutes at 300 g. The clarified supernatant was subsequently sterile filtered using a 0.22 μm vacuum filter and stored at 4° C. until use.

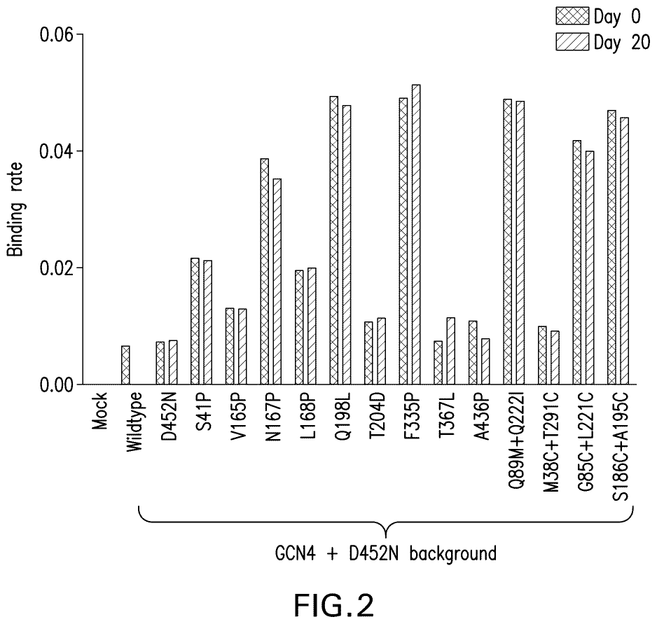

PIV3 F protein ectodomain was detected in crude supernatants using biolayer interferometry (BLI) measurements using quantitative Octet measurements with prefusion-specific monoclonal antibody PIA174 (Stewart-Jones et al., PNAS 115 (48) 12265-12270, 2018) immobilized to anti-human IgG sensors. While a low but distinct signal for wildtype (i.e. unstabilized) PIV3 preF protein was present at day of harvest (day 0), it was undetectable after 20 day storage at 4° C. (FIG. 2; wildtype).

### Example 2: Stabilizing Mutations Analyzed with Biolayer Interferometry and Analytical SEC

In order to stabilize the labile pre-fusion conformation of PIV3 F protein the ectodomain of PIV3 F was C-terminally fused to a GCN4 trimerization motif (SEQ ID NO: 3) and the amino acid residue Asp at position 452 was mutated into Asn (D452N). Next, additional mutations were introduced in this background as indicated in FIG. 2. Plasmids coding for the recombinant PIV3 F protein ectodomains equipped with a C-tag were expressed in Expi293Fcells, and 3 days after transfection the supernatants were tested for binding to PIA174 using quantitative Octet (FIG. 2). The variants showed binding to the prefusion trimer-specific Mab PIA174 at the day of harvest and the binding was maintained after storage at 4° C. for 20 days. Addition of the D452N mutation and GCN4 stabilized the prefusion conformation. The additional stabilizing mutations S41P, (Q89M+Q222I), V165P, N167P, L168P, Q198L, F335P, (S186C+A195C) and (G85C+L221C) increased the amount of prefusion PIV3 F protein in the crude cell supernatant, as compared to the construct with the D452N mutation only and also retained the prefusion conformation for 20 days in supernatant stored at 4° C.

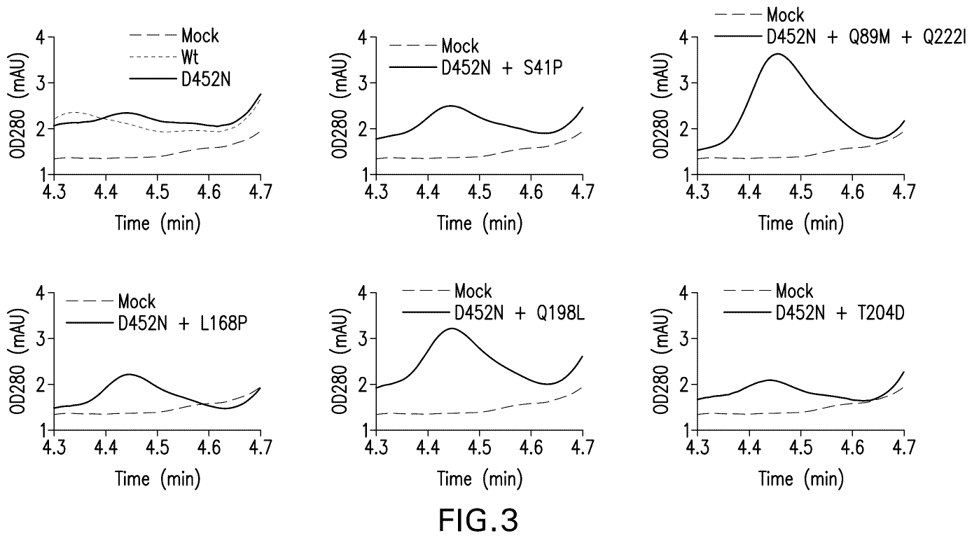

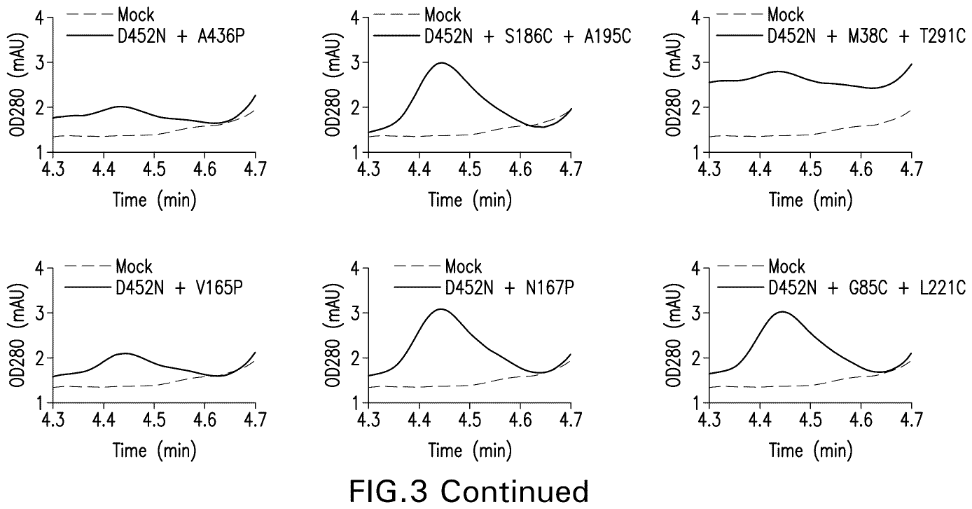

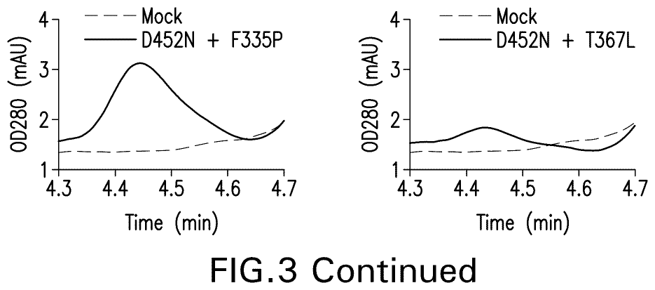

The cell culture supernatants of the different PIV3 F constructs with stabilizing mutations were analyzed using analytical size exclusion chromatography (SEC) (FIG. 3) at day of harvest. An ultra high-performance liquid chromatography system (Vanquish, Thermo Scientific) and μDAWN TREOS instrument (Wyatt) coupled to an Optilab μT-rEX Refractive Index Detector (Wyatt), in combination with an in-line Nanostar DLS reader (Wyatt), was used for performing the analytical SEC experiment. The cleared crude cell culture supernatants were applied to to a 300 Å column, (Sepax Cat #231300-4615) with the corresponding guard column (Sepax) equilibrated in running buffer (150 mM sodium phosphate, 50 mM NaCl, pH 7.0) at 0.35 mL/min. When analyzing supernatant samples, μMALS detectors were offline and analytical SEC data was analyzed using Chromeleon 7.2.8.0 software package. As was shown for the antibody binding studies described above, also the SEC analysis showed increase in trimer content upon introduction of the stabilizing mutations. Compared to either wildtype (SEQ ID NO: 2) or a variant with only GCN4 trimerization domain and D452N substitution (PIV171432; SEQ ID NO: 4), the variants with additional stabilizing substitutions showed higher trimer content according to analytical SEC of culture supernatant (FIG. 3). Compared with the soluble F variant with D452N and a C-terminal GCN4 domain, the variants with additional stabilizing substitutions S41P, (Q89M+Q2221), V165P, N167P, L168P, Q198L, F335P, (S186C+A195C) and (G85C+L221C) showed higher trimer content according to analytical SEC of culture supernatant (FIG. 3).

### Example 3: Analysis of Additive and Synergistic Stabilizing Mutations by Biolayer Interferometry

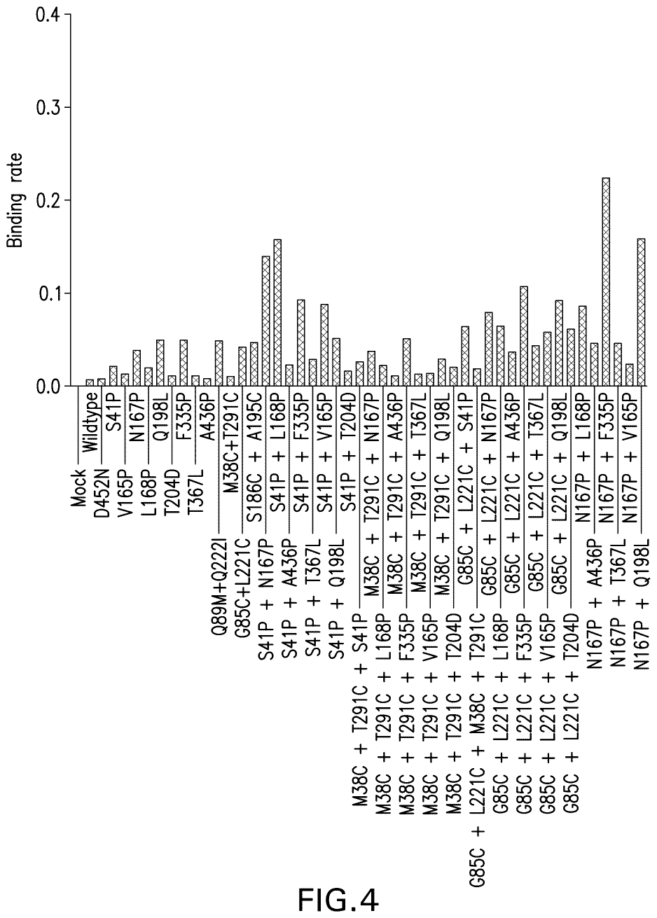

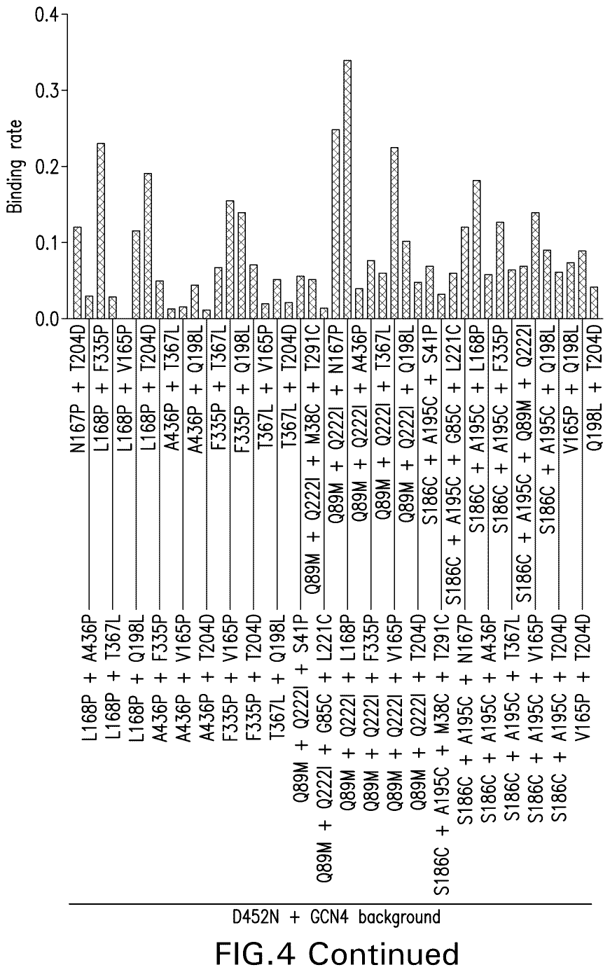

In order to further stabilize the labile pre-fusion conformation of PIV3 F protein ectodomains, constructs were made with additional mutations at amino acid residue positions 41, 89, 165, 167, 168, 198, 204, 222, 335, 367, and/or 436 in a D452N background (all constructs thus comprised the D452N mutation). Plasmids coding for these recombinant PIV3 F protein ectodomains which were C-terminally fused to a GCN4 (SEQ ID NO: 3) and equipped with a C-tag were expressed in Expi293Fcells, and 3 days after transfection the supernatants were tested for binding to PIA174 using quantitative Octet (FIG. 4). The variants showed binding to the prefusion trimer-specific Mab PIA174 at the day of harvest. Moreover, many of the double mutations had higher binding than each individual single mutation at position 41, 165, 167, 168, 198, 204, 335, 367 and 436 or the double mutation at 89+222 in the D452N background, indicating additive or even synergistic stabilizing effects.

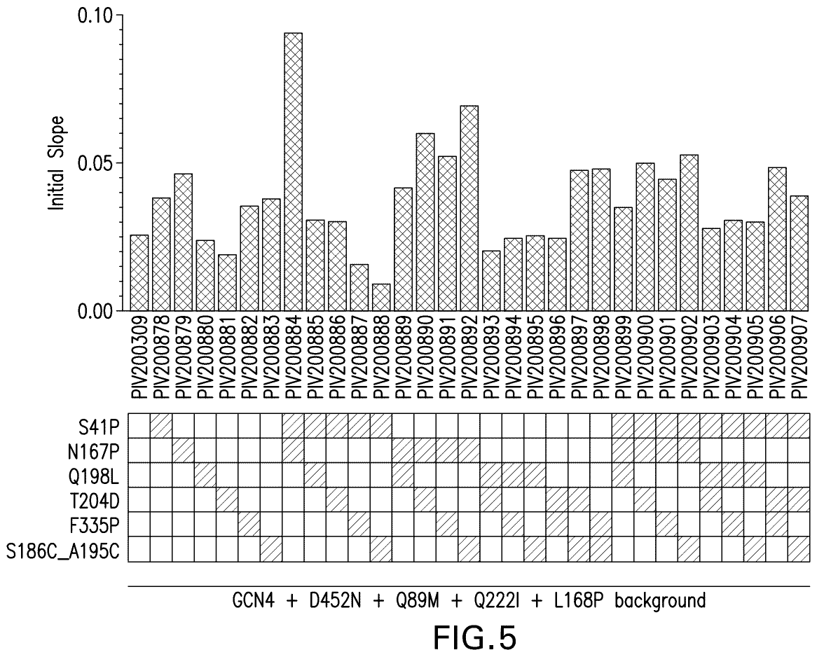

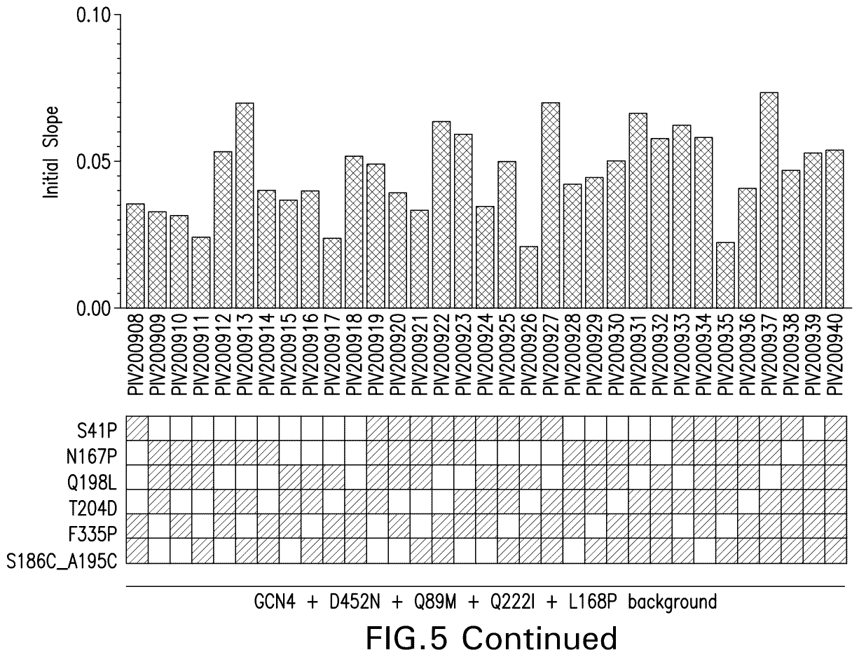

In order to further stabilize the PIV3 F protein ectodomain with the D452N+(Q89M+Q2221)+L168P mutations (i.e. PIV200309, SEQ ID NO:76) the previously mentioned stabilizing mutations were added in different combinations. Plasmids coding for these recombinant PIV3 F protein ectodomains which were C-terminally fused to a GCN4 and equipped with a C-tag were expressed in Expi293Fcells, and 3 days after transfection the supernatants were diluted 5-fold in mock transfected medium and tested for binding to PIA174 using quantitative Octet (FIG. 5). The variants showed binding to the prefusion trimer-specific Mab PIA174 at the day of harvest, with highest binding observed for PIV200884 (SEQ ID NO: 108) (Q89M/Q222I+L168P+S41P+N167P+D452N).

### Example 4: Stabilizing Mutations in the Stem Allow Removal of GCN4 Trimerization Domain while retaining trimer organization as analyzed with biolayer interferometry and analytical SEC

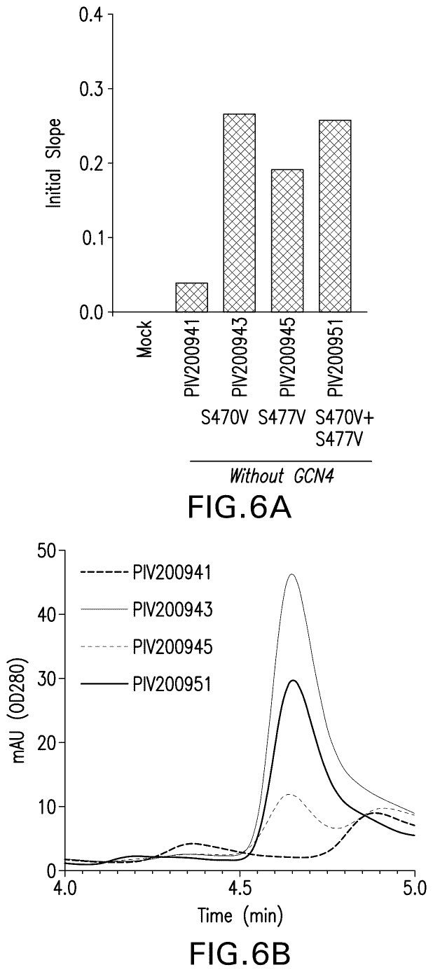

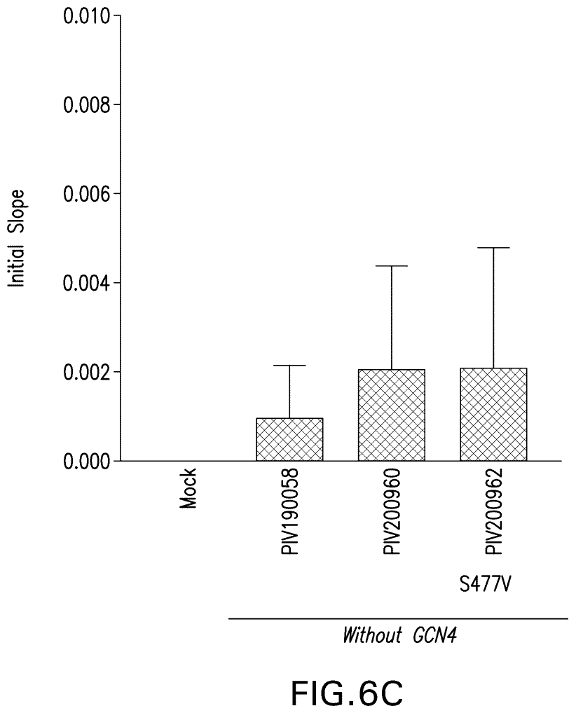

In order to stabilize the labile trimeric pre-fusion conformation of PIV3 F protein ectodomain in the absence of GCN4, amino acid residues at position 470 and 477 were mutated in the stem region (residues 452-481) of the PIV3 protein. Plasmids coding for a recombinant PIV3 F protein ectodomains which were equipped with a C-tag were expressed in Expi293Fcells, and 3 days after transfection the supernatants were tested for binding to PIA174 using quantitative Octet (FIG. 6A). The PIV3 backbone used was stabilized using D452N+Q89M+Q222I+L168P mutations without a GCN4 trimerization domain (PIV200941). Variants of PIV200941 that included 470V and/or 477V showed binding to the trimer-specific Mab PIA174, while a construct with wildtype amino acids at positions 470 and 477 showed only little prefusion trimer in supernatant. Therefore, addition of the 470V and 477V mutation stabilized the native trimeric quaternary structure of the prefusion F protein without a GCN4 trimerization domain.

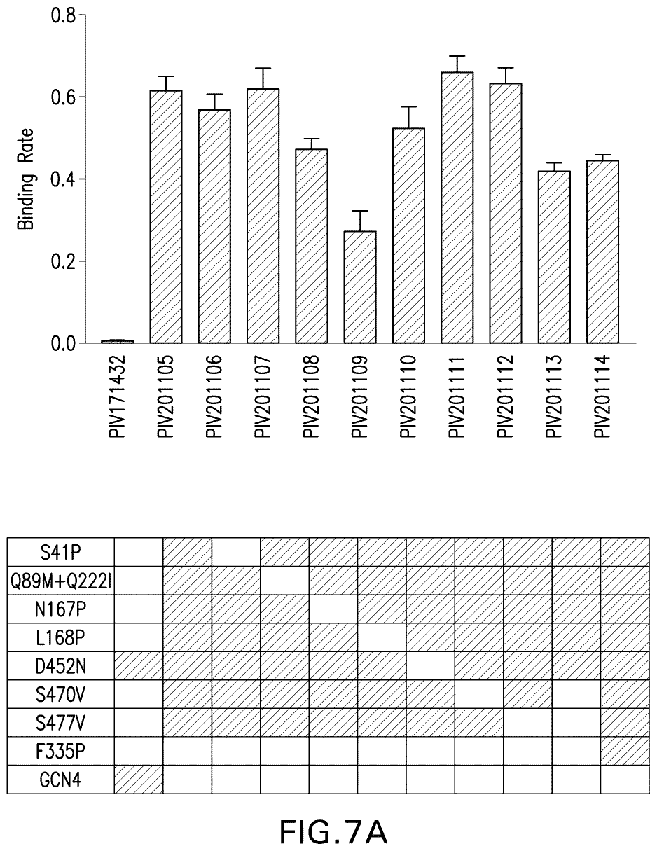

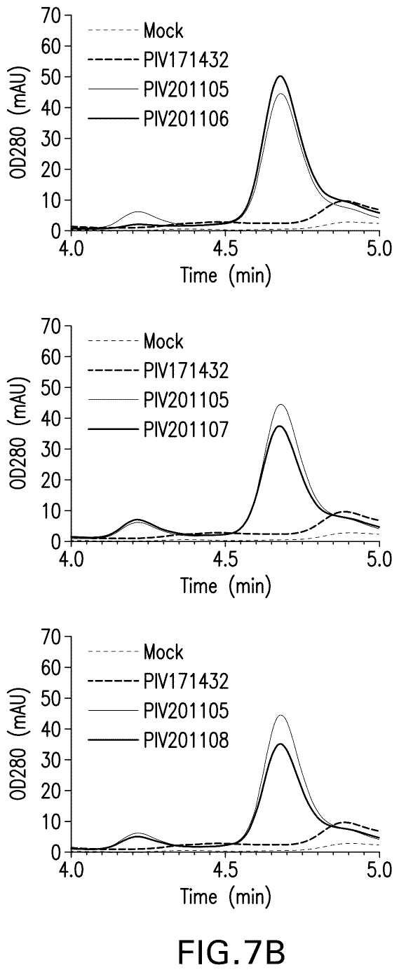

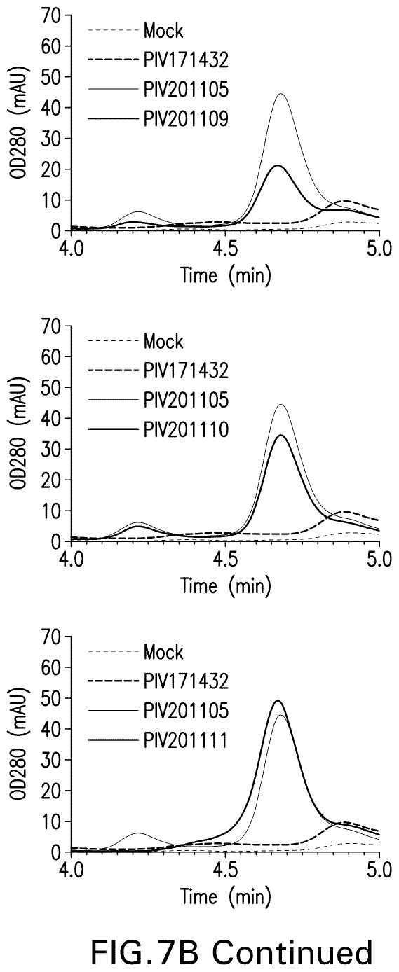

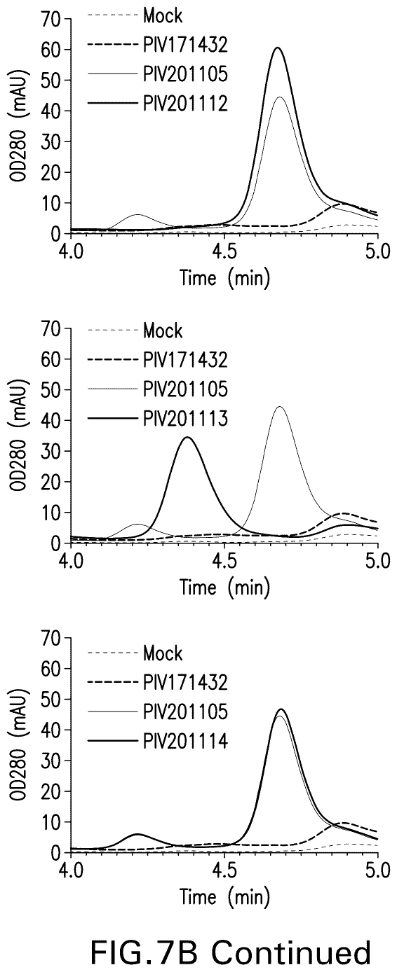

The cell culture supernatants of the different PIV3 F constructs with the 470V and/or 477V stabilizing mutations were analyzed using analytical size exclusion chromatography (SEC) (FIG. 7B) at day of harvest. Compared to a variant without stabilizing mutations in the stem and without GCN4 trimerization domain (PIV200941; comprising the D452N+Q89M+Q2221+L168P stabilizing mutations in the head domain), the variants with 470V and/or 477V stabilizing mutations showed higher trimer content according to analytical SEC of culture supernatant (FIG. 6B).

S470V and S477V were also studied in a wildtype backbone without GCN4 trimerization domain and without stabilizing mutations (PIV190058) (FIG. 6C). Introduction of S470V (PIV200960) or S477V (PIV200962) improved binding to PIA174, indicating that these mutations stabilize the pre-fusion conformation without any additional mutation or heterologous trimerization domain.

### Example 5: Combining Stabilizing Mutations in Head (Residues 19-451) and Stem (Residues

452-481) increases expression and stability of prefusion PIV3 F protein as determined by biolayer interferometry, analytical SEC and differential scanning fluorimetry.

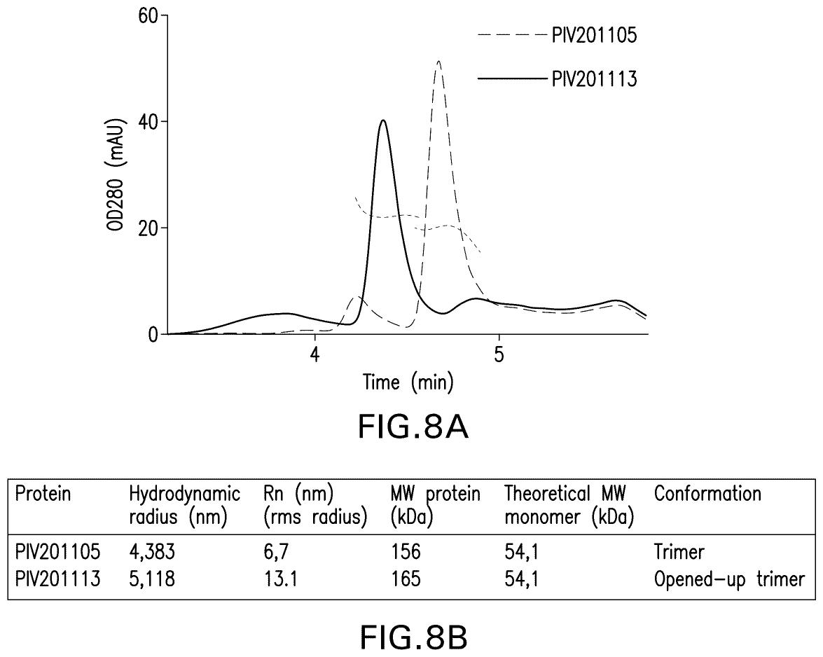

In order to stabilize the labile trimeric pre-fusion conformation of PIV3 F protein ectodomains in the absence of GCN4, the amino acid residues at position 41, 89, 167, 168, 222, 335, 452, 470 and/or 477 were mutated. Plasmids coding for the recombinant PIV3 F protein ectodomains which were equipped with a C-tag were expressed in Expi293Fcells, and 3 days after transfection the supernatants were tested for binding to PIA174 using quantitative Octet (FIG. 7A) and analytical SEC (FIG. 7B). In the absence of 470V and 477V the protein runs at a lower retention time, indicating that the protein is larger. MALS analysis of PIV201113 and PIV201105 (FIG. 8) shows that there is no significant difference in molecular weight (165 vs 156 kDa, respectively). The lower retention time of PIV201113 is presumably because of opening up of the stem region, increasing the proteins apparent size which will be partly trimerized at the apex but not as compact as PIV201105 which will also be trimerized at the base by the optimized stem mutations. The amino acids 470V and/or 477V are thus required to keep the protein in a native trimeric conformation in absence of a heterologous trimerization domain.

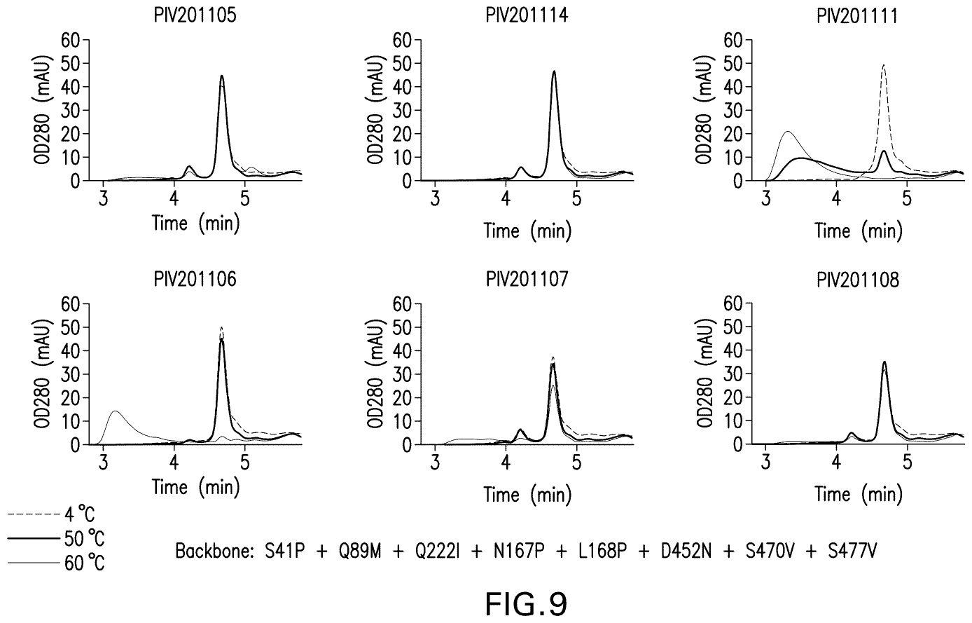

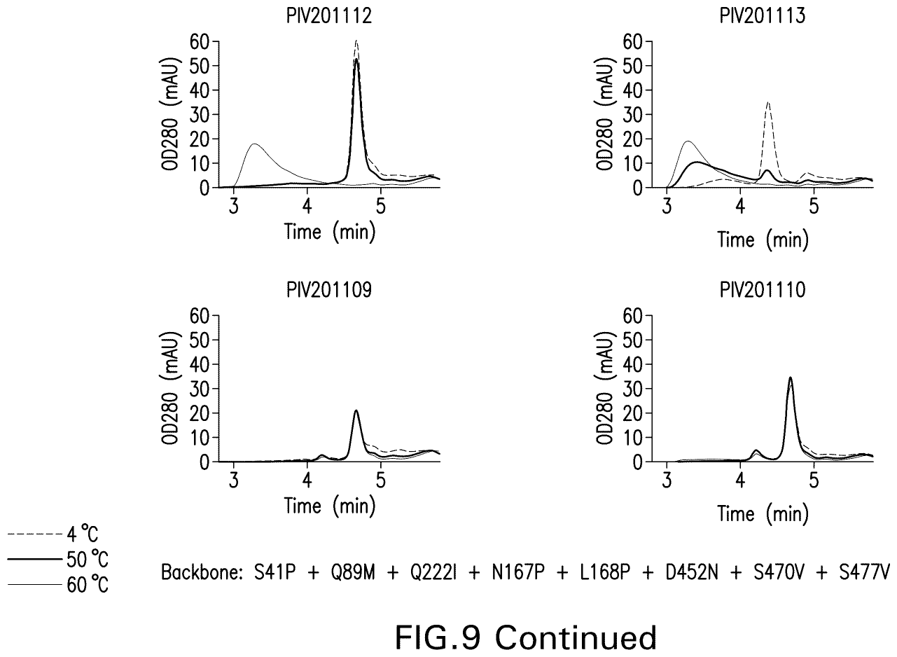

In addition, the stability of the different proteins in supernatant was determined by incubating the samples at 4° C., 50° C. or 60° C. for 30 minutes in a heat block. The samples were then spun at 15.000 rpm for 10 minutes to remove larger aggregates and the supernatant was run on analytical SEC (FIG. 9). Without 470V the protein already loses trimer conformation at 50° C. Without 477V the protein loses trimer conformation at 60° C. Absence of 41P also reduces stability of the protein as the trimer peak is completely gone after 30 min incubation at 60° C.

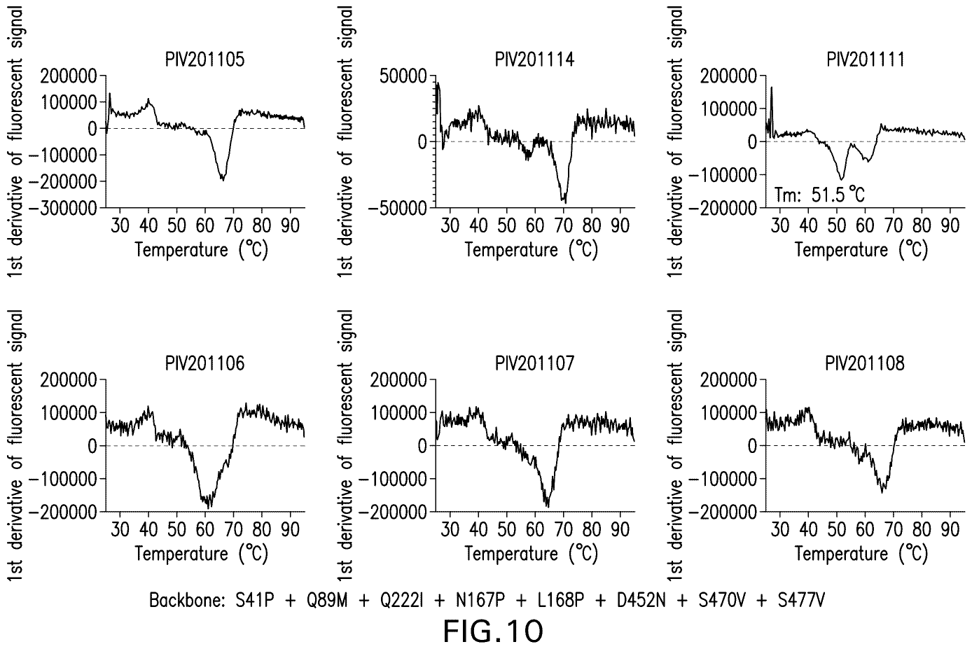

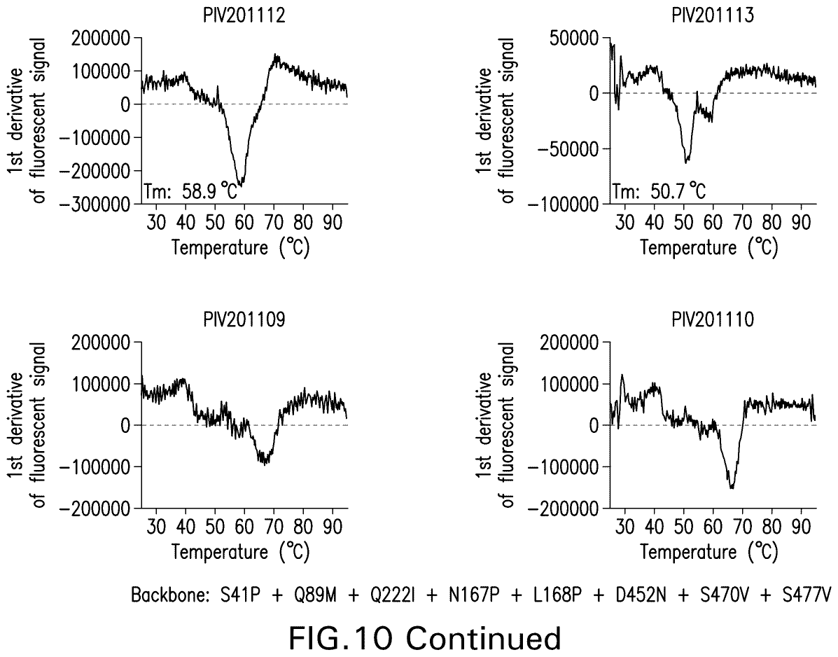

Stability of the different proteins in supernatant was also determined by measuring the melting temperature (Tm) using differential scanning fluorimetry (DSF). To this end, SYPRO Orange 5000x (S6650, Invitrogen) was diluted in PBS (1:250) to obtain a 20× working solution. For each reaction, 15 μL of the supernatant was mixed with 5 μL of the SYPRO 20× in a MicroAmp Fast Optical 96-well plate (4346906, ThermoFisher). PBS was used as a negative control. The plate was covered with a MicroAmp Optical Adhesive Film (4311971, ThermoFisher) and was subsequently read in a ViiA7 Real-time PCR machine. The construct with all stabilizing mutations (S41P+Q89M+Q222I+N167P+L168P+D452N+S470V+S477V+F335P) and without GCN4 had a Tm50 of 70.7° C. (′Backbone+F335P′ FIG. 10).

Removal of 335P reduced the Tm50 to 66.4° C. (FIG. 10). Additional removal of 470V and/or 477V drastically reduced the Tm50 to <59° C. Further removal of 41P or 89M+222I reduced the Tm to 61.1° C. and 64.7° C., respectively. This indicates that especially S470V and S477V are required for a stable soluble trimer in the absence of a GCN4 trimerization domain. In addition, S41P, F335P and Q89M+Q222I increase the melting temperature and are thus further stabilizing the protein.

### Example 6: Characterization of Purified PIV3 F Protein as Determined by SDS-PAGE, Analytical SEC and Differential Scanning Fluorimetry

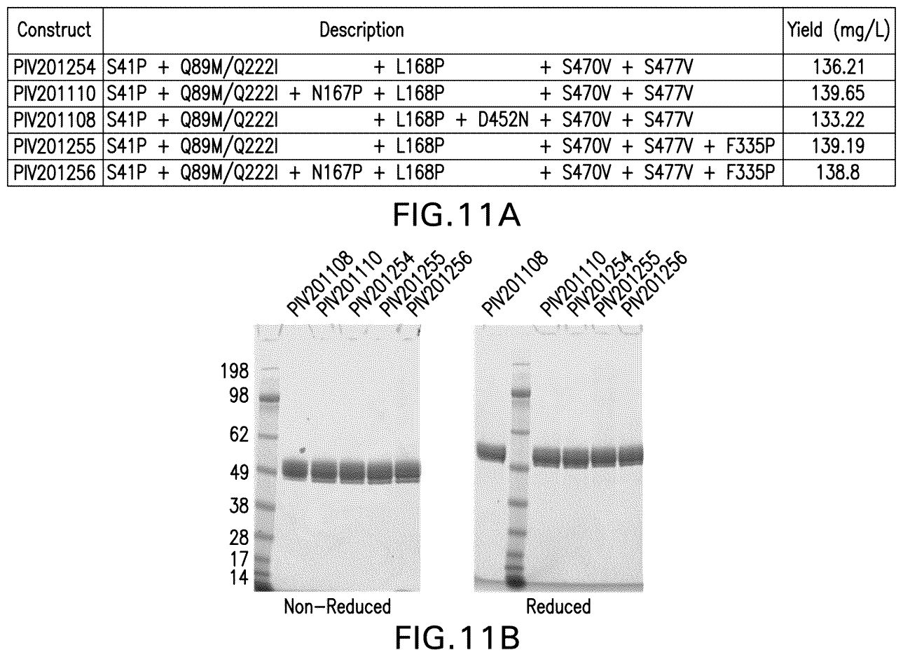

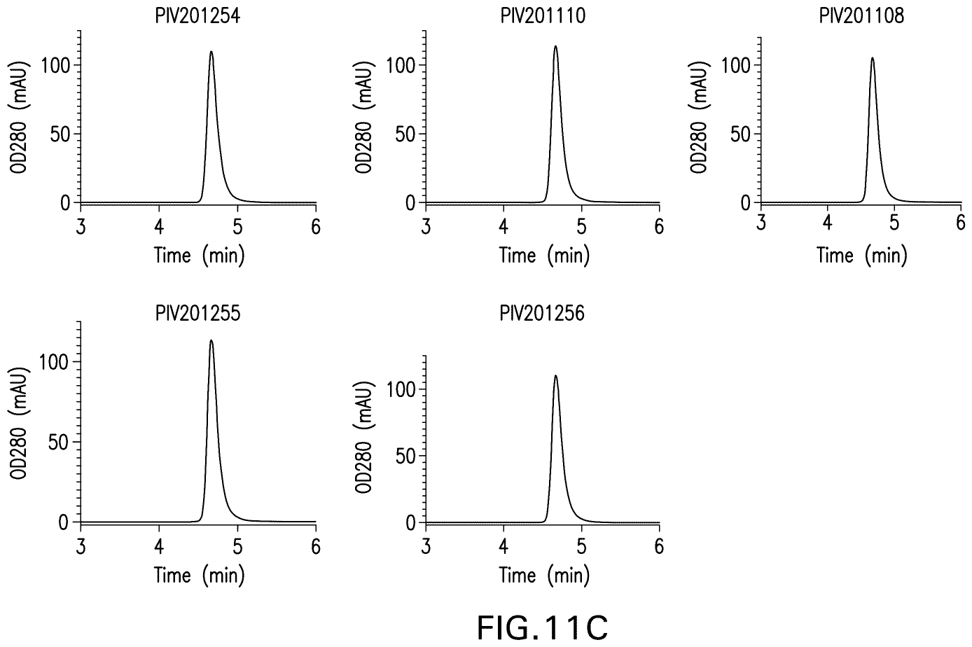

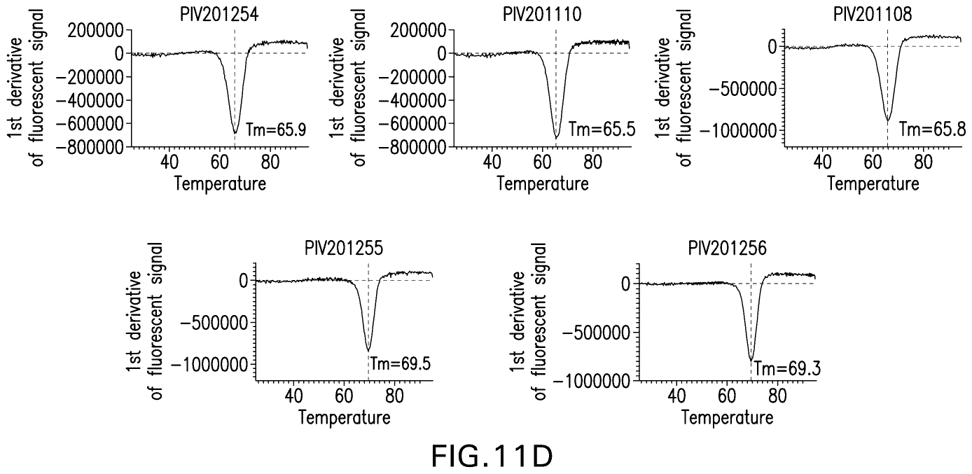

A set of PIV3 F designs (overview in FIG. 11A) was transiently transfected in expi293 cells using ExpiFectamine (Life Technologies) according to the manufacturer's instructions and cultured for 5 days at 370C and 10% CO2. The culture supernatant was harvested and spun for 10 minutes at 600 g to remove cells and cellular debris. The spun supernatant was subsequently sterile filtered using a 0.22 μm vacuum filter and stored at 4° C. until use. PIV3 F proteins were purified using a two-step purification protocol including CaptureSelect™ C-tag affinity column, followed by size-exclusion chromatography using a HiLoad Superdex200 μg 16/600column (GE Healthcare).

Yield for each protein design in mg/L after purification are indicated in FIG. 11A. Purified proteins were analyzed under reducing and non-reducing conditions on SDS-PAGE and developed with Coomassie (FIG. 11B). The proteins ran as a single, non-processed, band, indicating that cleavage at the F2/F1 boundary does not take place in expiHEK cells. SEC-MALS showed a clean trace with a sharp peak at the size of the PIV3 F trimer (FIG. 11C). Differential scanning fluorimetry (DSF) showed that designs that include F335P (PIV201255 and PIV201256) show a 3.6-3.8° C. increase in melting temperature compared to their counterparts without F335P (PIV201254 and PIV201110, respectively) (FIG. 11D).

### Example 7: Stabilizing Mutations S470V+S477V in the HR2 Stem are Sufficient for Trimer Formation as Analyzed with Biolayer Interferometry and Analytical SEC

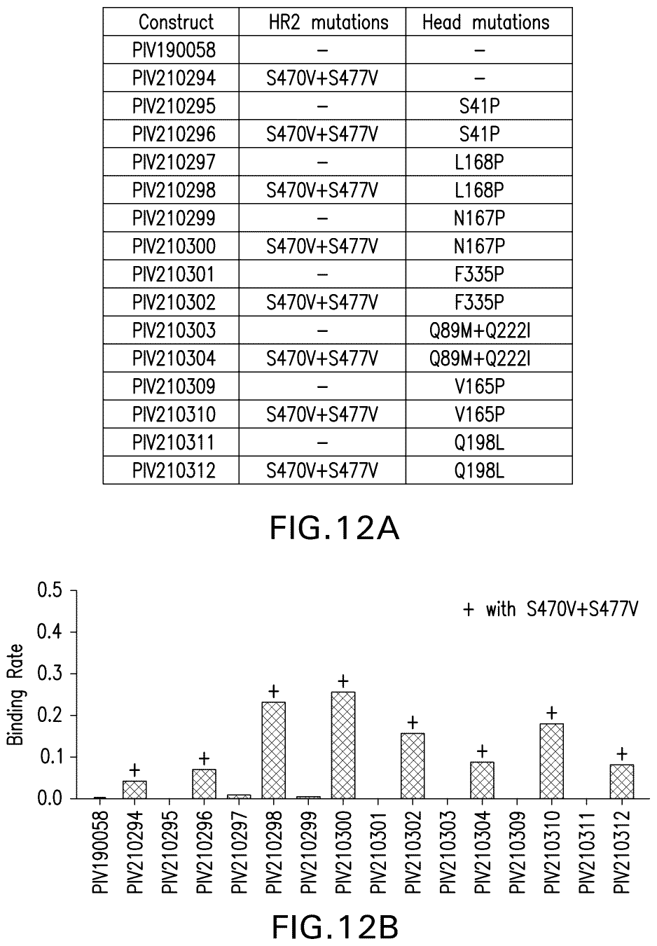

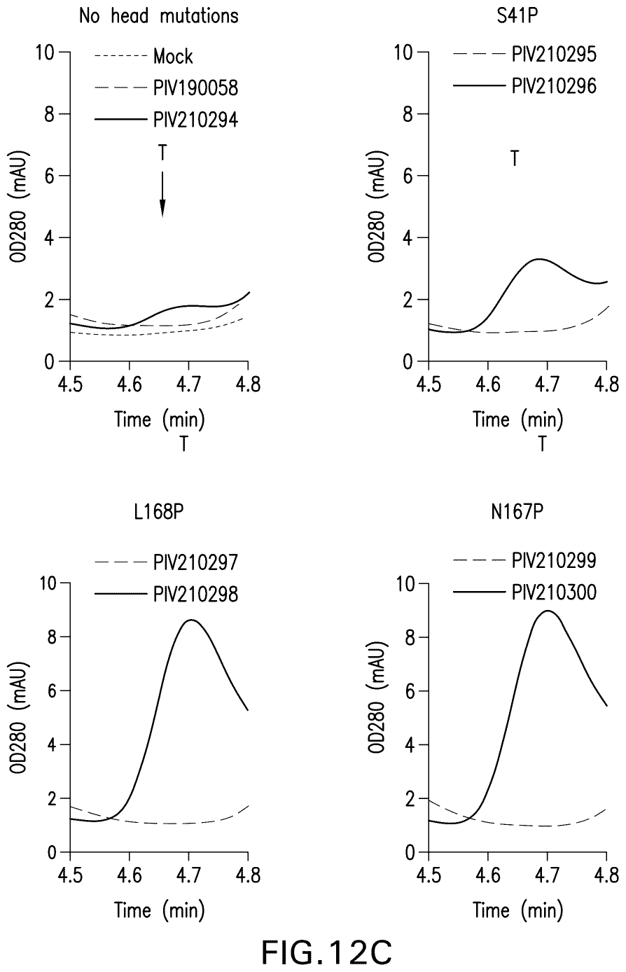

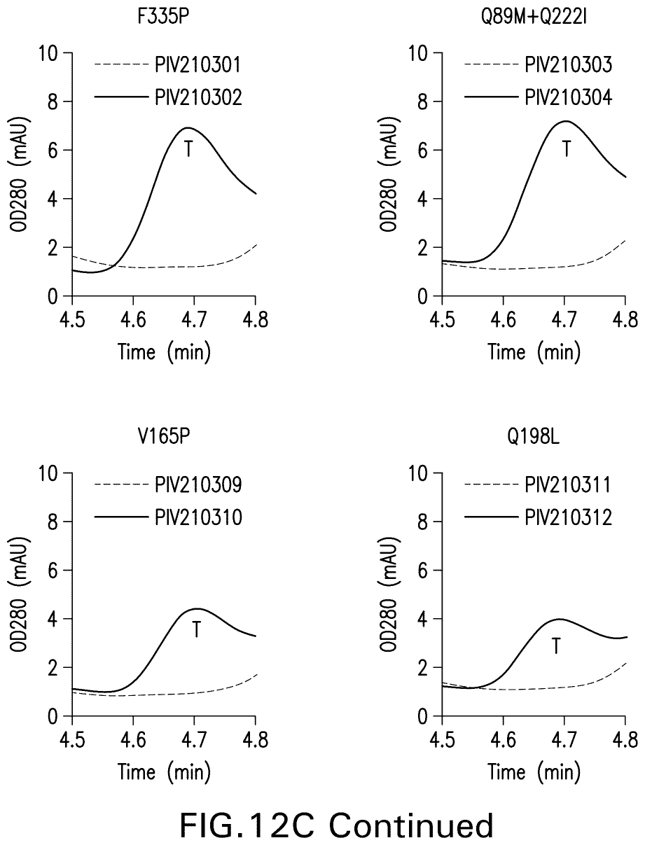

In order to stabilize the labile trimeric pre-fusion conformation of PIV3 F protein ectodomain in the absence of GCN4, amino acid residues at position 470 and 477 were mutated in the stem region (residues 452-481) of the PIV3 protein. Plasmids coding for a recombinant PIV3 F protein ectodomains which were equipped with a C-tag were expressed in Expi293Fcells, and 3 days after transfection the supernatants were tested for binding to PIA174 using quantitative Octet as described in example 1 (FIG. 12B) and were analyzed for trimer content using analytical size exclusion chromatography (SEC) as described in example 2 (FIG. 12C). A wildtype PIV3 backbone (no stabilizing mutations, no GCN4 trimerization domain; PIV190058) showed no binding to the trimer-specific Mab PIA174 (FIG. 12B) nor a detectable trimer peak in supernatant (FIG. 12C). In contrast, PIA174 Mab binding and a detectable trimer peak was observed upon introduction of the S470V and S477V HR2 stem mutations (PIV210294), demonstrating that addition of the 470V and 477V mutation stabilized the native trimeric quaternary structure of the prefusion F protein without a GCN4 trimerization domain. Subsequent introduction of various head domain mutations (FIG. 12A) increased PIA174 binding in quantitative Octet and improved trimer yield in analytical SEC, compared to S470V+S477V alone. However, head domain mutations alone did not yield detectable trimer binding and trimer expression in supernatant (FIG. 12C, dotted lines), underscoring the importance of HR2 stabilization for the native trimeric quaternary structure of soluble PIV3 prefusion F (FIG. 12C, solid lines).

### Example 8: Contribution of Individual Mutations to the Stability and Yield of PIV3 Prefusion F Design PIV211368

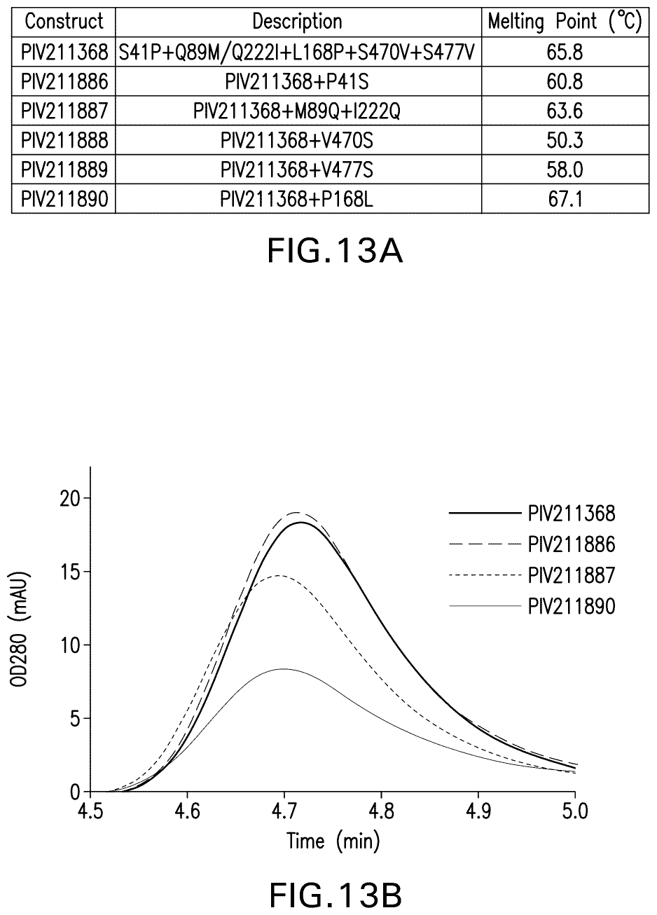

Expression of PIV3 F protein including head stabilizing mutations S41P, Q89M+Q222I, and L168P and stem stabilizing mutations S470V+S477V (PIV211368) was compared to PIV3 F variants in which single or double mutations were systematically removed by reverting the amino acid to wildtype (indicated in bold in FIG. 13A). Plasmids coding for the recombinant PIV3 F protein ectodomains without a purification tag were expressed in Expi293F cells, and 3 days after transfection the stability of the different proteins in supernatant was determined by measuring the melting temperature (Tm50) using differential scanning fluorimetry (DSF) as described in example 5 (FIG. 13A), and trimer content was assessed in analytical SEC as described in example 2 (FIG. 13B). Head domain mutations that contribute to stability are S41P and Q98M+Q1221, as they have lower melting temperature (respectively 60.8° C. and 63.6° C.) than PIV211368 (65.8° C.) when reverted to wildtype. HR2 mutations S470V and S477V similarly contribute to PIV3 F temperature stability, with lower melting temperatures of 50.3° C. and 58.0° C., respectively. In contrast, head domain L168P substitution decreases thermal stability, as shown by an increased melting temperature of 67.1° C. when reverted to wildtype (FIG. 13A).

Head domain mutations either have little to no impact on PIV3 F trimer content (P41S; PIV211886) or have a positive impact (M89Q+1222Q; PIV211887 and P168L; PIV211890), as demonstrated by decreased trimer peak of wildtype-reverted variants (FIG. 13B).

In conclusion, in this particular stabilized protein design HR2 substitutions S470V and S477V strongly contribute to PIV3 F protein stability, whereas head domain mutation L168P strongly contributes to trimer expression but not to protein stability. Head domain mutations S41P and Q89M+Q222I contribute to thermal stability and the latter combination also increases trimer yield.

### Example 9: Characterization of Purified Tag-Less PIV3 F Protein as Determined by Analytical SEC, Differential Scanning Fluorimetry and Slow-Freeze Stability

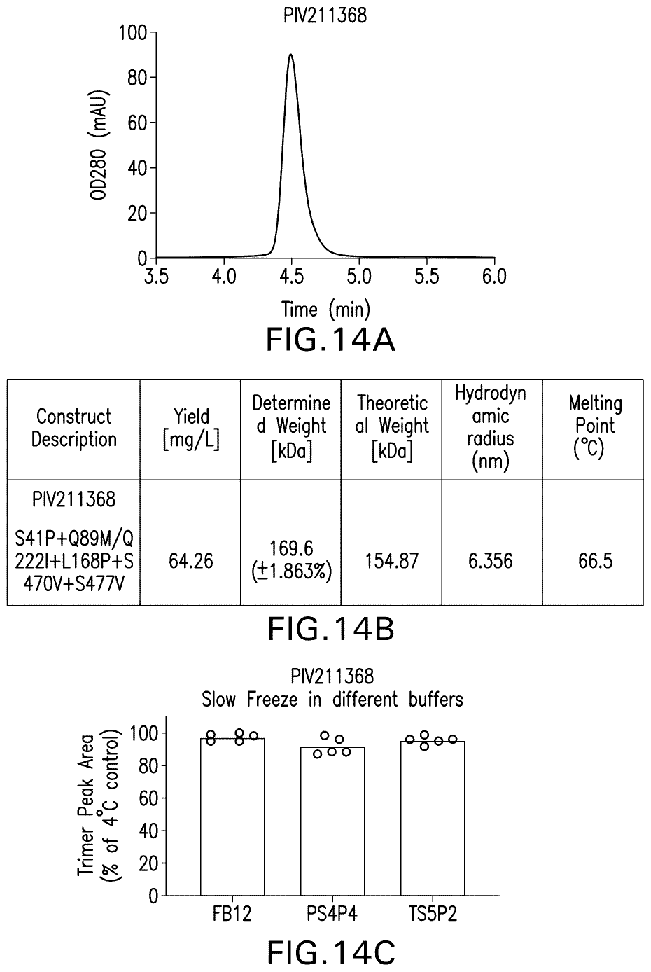

PIV3 F design PIV211368 without a purification tag and with stabilizing mutations S41P, Q89M/Q222I, L168P, S470V, and S477V was transiently transfected in Expi293F cells using ExpiFectamine (Life Technologies) according to the manufacturer's instructions and cultured for 5 days at 37° C. and 10% CO2. The culture supernatant was harvested and spun for 10 minutes at 600 g to remove cells and cellular debris. The spun supernatant was subsequently sterile filtered using a 0.22 μm vacuum filter and stored at 4° C. until use. PIV3 F protein was purified using a two-step purification protocol including ion exchange purification at pH 4.0 and polishing via size exclusion chromatography using a Superdex 200 increase 16/40 column. The trimeric fraction was pooled and further characterized by SEC-MALS (FIG. 14A). Trimer yield, molecular weight and hydrodynamic radius is reported in FIG. 14B. Differential scanning fluorimetry (DSF) showed that purified PIV211368 has a melting temperature of 66.5° C. (FIG. 14B) which is slightly higher compared with the measurement of the protein in crude cell culture supernatant (FIG. 13A). The stability of purified PIV211368 was further tested by slowly freezing the protein from 20° C. to −70° C. during a 24-hour period in various buffer compositions (FB12, PS4P4 and TS5P2). The recovery of PIV3 F trimer after slow freezing was determined in analytical SEC and compared to trimer recovery after 4° C. storage. Recovery ranged from 92-98%, indicating minimal trimer loss in any of the tested buffers.

### Example 10: Characterization of Purified C-Tagged PIV3 F Protein as Determined by Analytical SEC and Differential Scanning Fluorimetry

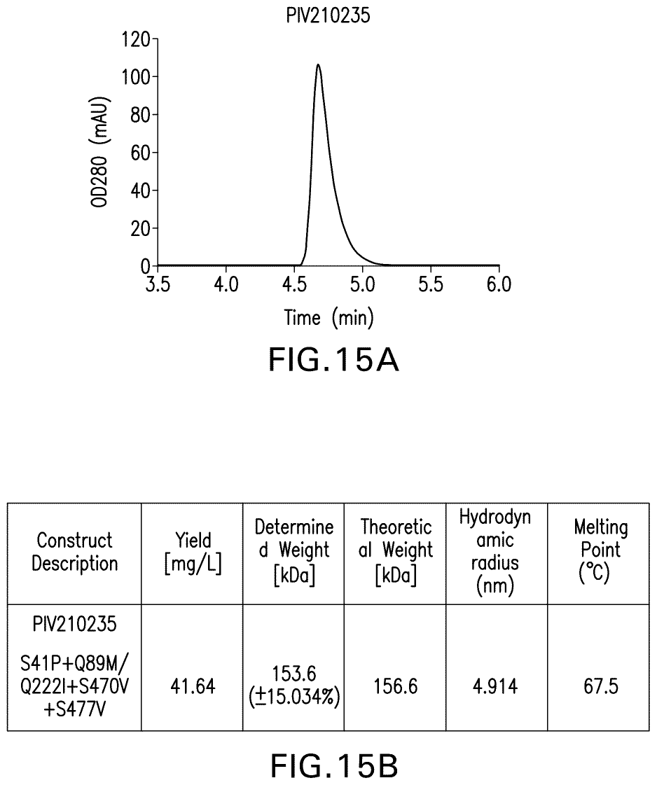

PIV3 F design PIV210235 equipped with a C-tag and with stabilizing mutations S41P, Q89M/Q2221, S470V, and S477V was transiently transfected in Expi293 GnT1− cells using ExpiFectamine (Life Technologies) according to the manufacturer's instructions and cultured for 5 days at 37° C. and 10% CO2. The culture supernatant was harvested and spun for 10 minutes at 600 g to remove cells and cellular debris. The spun supernatant was subsequently sterile filtered using a 0.22 μm vacuum filter and stored at 4° C. until use. PIV3 F protein was purified using a two-step purification protocol including CaptureSelect™ C-tag affinity column, followed by size-exclusion chromatography using a Superdex200 10/300 column (GE Healthcare). The trimeric fraction was pooled and further characterized by SEC-MALS (FIG. 15A). Trimer yield, molecular weight and hydrodynamic radius is reported in FIG. 15B. Differential scanning fluorimetry (DSF) showed that purified PIV210235 has a melting temperature of 67.5° C. (FIG. 15B).

### Example 11: Fully Single Chain Proteins According to the Invention

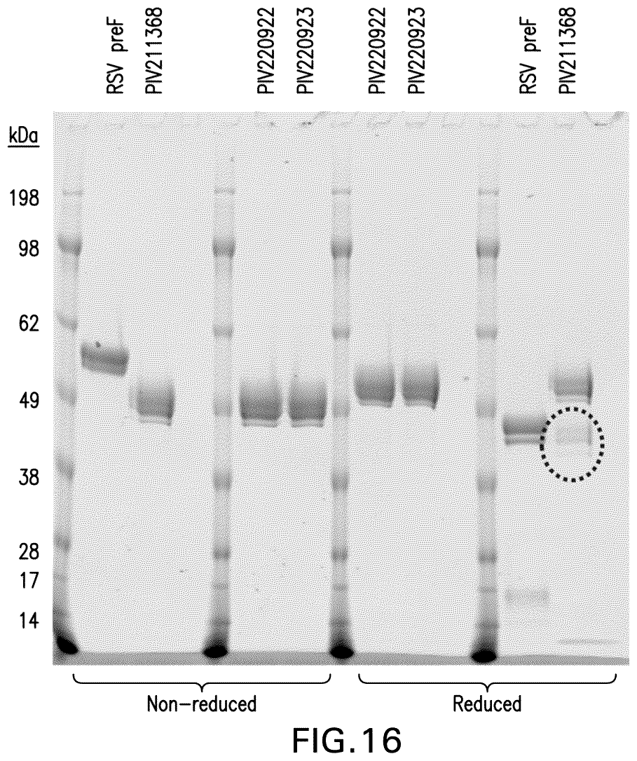

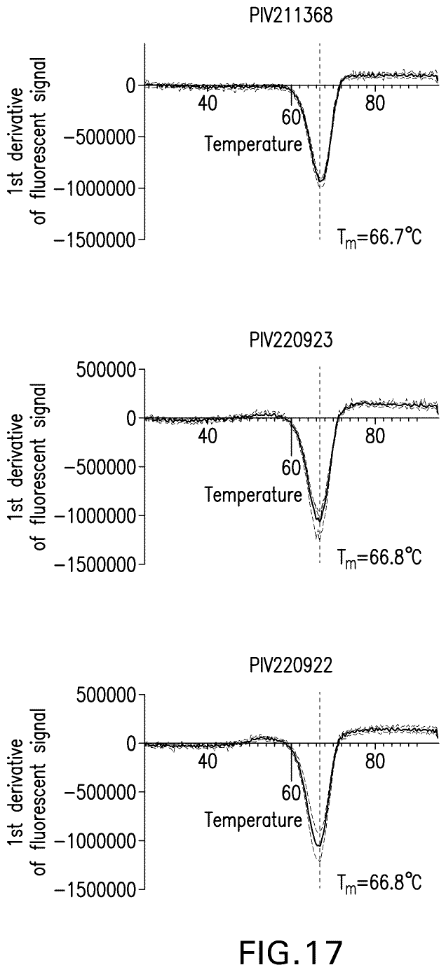

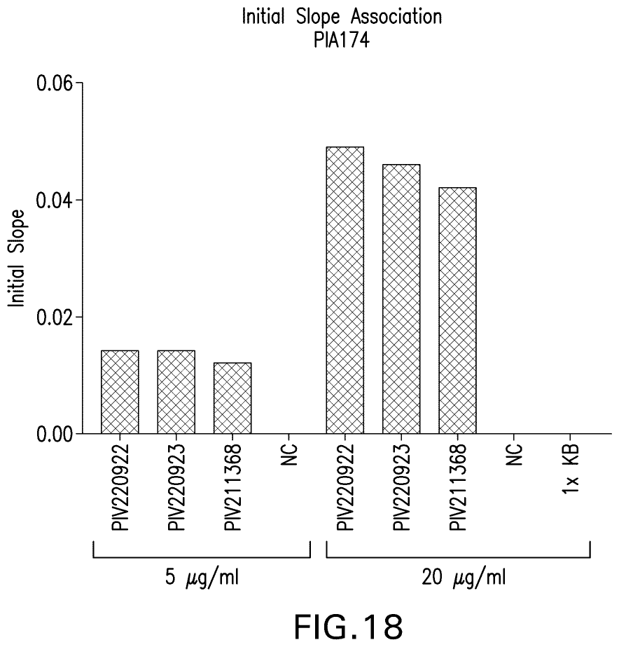

Purification (IEX, followed by SEC) of PIV211368 (SEQ ID NO: 237) resulted in a protein that was unexpectedly partially processed into F2 and F1 (even though the cleavage site (‘RTER’) is not recognized by furin-linke proteases, and the native protease TMPRSS2 is not expressed in expiHEK cells) as detected on reduced SDS-PAGE followed by Coomassie staining (FIG. 16). Introduction of R109Q (and T95A) resulted in a fully single-chain protein after IEX/SEC purification. Introduction of the E58D mutation did not affect the processing (compare PIV220923 to PIV220922). All three proteins show similar thermal stability, as determined by DSF (FIG. 17). Purified PIV220922 and PIV220923 proteins show slightly higher binding to PIV3 prefusion-specific antibody PIA174 (FIG. 18).

TABLE 1
Standard amino acids, abbreviations and properties

| | | | | Side chain |
|---|---|---|---|---|
| | | | Side chain | charge |
| Amino Acid | 3-Letter | 1-Letter | polarity | (pH 7.4) |
| alanine | Ala | A | non-polar | Neutral |
| arginine | Arg | R | polar | Positive |
| asparagine | Asn | N | polar | Neutral |
| aspartic acid | Asp | D | polar | Negative |
| cysteine | Cys | C | non-polar | Neutral |
| glutamic acid | Glu | E | polar | Negative |
| glutamine | Gln | Q | polar | Neutral |
| glycine | Gly | G | non-polar | Neutral |
| histidine | His | H | polar | positive(10%) |
| | | | | neutral(90%) |
| isoleucine | Ile | I | non-polar | Neutral |
| leucine | Leu | L | non-polar | Neutral |
| lysine | Lys | K | polar | Positive |
| methionine | Met | M | non-polar | Neutral |
| phenylalanine | Phe | F | non-polar | Neutral |
| proline | Pro | P | non-polar | Neutral |
| Serine | Ser | S | polar | Neutral |
| Threonine | Thr | T | polar | Neutral |
| Tryptophan | Trp | W | non-polar | Neutral |
| tyrosine | Tyr | Y | polar | Neutral |
| valine | Val | V | non-polar | Neutral |

SEQUENCES

SEQ ID NO: 1: PIV191469 (full-length, membrane-bound HPIV3 F)
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGSWHQSSTTIIVILIMMIIL
FIINITIITIAIKYYRIQKRDRVDQNDKPYVLTNK
SEQ ID NO: 2: PIV190058 (Wildtype ectodomain, no GCN4, but with
a C-tag)
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGGSEPEA
SEQ ID NO: 3: GCN4
EDKIEEILSKIYHIENEIARIKK
SEQ ID NO: 4 PIV171432
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 5 PIV190059
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIGGSEPEA
SEQ ID NO: 6 PIV191486
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 7 PIV191489
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLLLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 8 PIV191501
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 9 PIV191523
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDVSIELNKVKSDLEEVKEWLRRVNQKLDSIGSGEPEA
SEQ ID NO: 10 PIV191528
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDVSIELNKVKSKLEDVKEWLRRVNQKLDSIGSGEPEA
SEQ ID NO: 11 PIV191530
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKVKSDLEEVKEWLRRVNQKLDSIGSGEPEA
SEQ ID NO: 12 PIV191531
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKIKSDLEEIKEWLRRVNQKLDSIGSGEPEA
SEQ ID NO: 13 PIV191551
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 14 PIV191557
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 15 PIV191560
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGPLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 16 PIV191561
MPISILLIITTMIMASHCQIDITKLQHVGVLVNSPKGMKISQNFETRYLILSLIPKIEDSNSC
GDQQIKQYKRLLDRLIIPLYDCLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSCQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 17 PIV191580
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 18 PIV191593
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGSGEPEA
SEQ ID NO: 19 PIV191594
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIGSGEPEA
SEQ ID NO: 20 PIV200253
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGPLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 21 PIV200254
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 22 PIV200255
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLPFYTPD
DITLNNSVALNPIDISIELNKAKSDLEESKEWIRRSNQKLDSIEDKIEEILSKIYHIENEIAR
IKKLIGEAEPEA
SEQ ID NO: 23 PIV200256
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 24 PIV200257
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTLVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 25 PIV200258
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 26 PIV200259
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLLLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 27 PIV200260
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALDQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 28 PIV200261
MPISILLIITTMIMASHCQIDITKLQHVGVLVNSPKGCKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNCQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 29 PIV200262
MPISILLIITTMIMASHCQIDITKLQHVGVLVNSPKGCKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGPLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNCQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 30 PIV200263
MPISILLIITTMIMASHCQIDITKLQHVGVLVNSPKGCKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNCQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 31 PIV200264
MPISILLIITTMIMASHCQIDITKLQHVGVLVNSPKGCKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNCQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLPFYTPD
DITLNNSVALNPIDISIELNKAKSDLEESKEWIRRSNQKLDSIEDKIEEILSKIYHIENEIAR
IKKLIGEAEPEA
SEQ ID NO: 32 PIV200265
MPISILLIITTMIMASHCQIDITKLQHVGVLVNSPKGCKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNCQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 33 PIV200266
MPISILLIITTMIMASHCQIDITKLQHVGVLVNSPKGCKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNCQIYKVDSISYNIQNREWYIPLPSH
IMTKGAFLGGADVKECIEAFSSYICPSDPGFVLNHEMESCLSGNISQCPRTLVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 34 PIV200267
MPISILLIITTMIMASHCQIDITKLQHVGVLVNSPKGCKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNCQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 35 PIV200268
MPISILLIITTMIMASHCQIDITKLQHVGVLVNSPKGCKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLLLGIALTQHYSELTNIFGDNIGSLQEKGIKLQGIASLYRTNITEIFTTSTVDKYD
IYDLLFTESIKVRVIDVDLNDYSITLQVRLPLLTRLLNCQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 36 PIV200269
MPISILLIITTMIMASHCQIDITKLQHVGVLVNSPKGCKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALDQHYSELTNIFGDNIGSLQEKGIKLQGIASLYRTNITEIFTTSTVDKYD
IYDLLFTESIKVRVIDVDLNDYSITLQVRLPLLTRLLNCQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 37 PIV200270
MPISILLIITTMIMASHCQIDITKLQHVGVLVNSPKGMKIPQNFETRYLILSLIPKIEDSNSC
GDQQIKQYKRLLDRLIIPLYDCLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSCQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 38 PIV200271
MPISILLIITTMIMASHCQIDITKLQHVGVLVNSPKGCKISQNFETRYLILSLIPKIEDSNSC
GDQQIKQYKRLLDRLIIPLYDCLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSCQEKGIKLQGIASLYRTNITEIFTTSTVDKYD
IYDLLFTESIKVRVIDVDLNDYSITLQVRLPLLTRLLNCQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 39 PIV200272
MPISILLIITTMIMASHCQIDITKLQHVGVLVNSPKGMKISQNFETRYLILSLIPKIEDSNSC
GDQQIKQYKRLLDRLIIPLYDCLRLQKDVIVTNQESNENTDPRTERFFGGVIGTIALGVATSA
QITAAVALVEAKQARSDIEKLKEAIRDTNKAVQSVQSSVGPLIVAIKSVQDYVNKEIVPSIAR
LGCEAAGLQLGIALTQHYSELTNIFGDNIGSCQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 40 PIV200273
MPISILLIITTMIMASHCQIDITKLQHVGVLVNSPKGMKISQNFETRYLILSLIPKIEDSNSC
GDQQIKQYKRLLDRLIIPLYDCLRLQKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSCQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 41 PIV200274
MPISILLIITTMIMASHCQIDITKLQHVGVLVNSPKGMKISQNFETRYLILSLIPKIEDSNSC
GDQQIKQYKRLLDRLIIPLYDCLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSCQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLPFYTPD
DITLNNSVALNPIDISIELNKAKSDLEESKEWIRRSNQKLDSIEDKIEEILSKIYHIENEIAR
IKKLIGEAEPEA
SEQ ID NO: 42 PIV200275
MPISILLIITTMIMASHCQIDITKLQHVGVLVNSPKGMKISQNFETRYLILSLIPKIEDSNSC
GDQQIKQYKRLLDRLIIPLYDCLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSCQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 43 PIV200276
MPISILLIITTMIMASHCQIDITKLQHVGVLVNSPKGMKISQNFETRYLILSLIPKIEDSNSC
GDQQIKQYKRLLDRLIIPLYDCLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSCQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTLVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 44 PIV200277
MPISILLIITTMIMASHCQIDITKLQHVGVLVNSPKGMKISQNFETRYLILSLIPKIEDSNSC
GDQQIKQYKRLLDRLIIPLYDCLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPSIAR
LGCEAAGLQLGIALTQHYSELTNIFGDNIGSCQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 45 PIV200278
MPISILLIITTMIMASHCQIDITKLQHVGVLVNSPKGMKISQNFETRYLILSLIPKIEDSNSC
GDQQIKQYKRLLDRLIIPLYDCLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLLLGIALTQHYSELTNIFGDNIGSCQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 46 PIV200279
MPISILLIITTMIMASHCQIDITKLQHVGVLVNSPKGMKISQNFETRYLILSLIPKIEDSNSC
GDQQIKQYKRLLDRLIIPLYDCLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALDQHYSELTNIFGDNIGSCQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 47 PIV200280
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 48 PIV200281
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGPLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLPFYTPD
DITLNNSVALNPIDISIELNKAKSDLEESKEWIRRSNQKLDSIEDKIEEILSKIYHIENEIAR
IKKLIGEAEPEA
SEQ ID NO: 49 PIV200282
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGPLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 50 PIV200283
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGPLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTLVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 51 PIV200284
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGPLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 52 PIV200285
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGPLIVAIKSVQDYVNKEIVPSIAR
LGCEAAGLLLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 53 PIV200286
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGPLIVAIKSVQDYVNKEIVPSIAR
LGCEAAGLQLGIALDQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 54 PIV200287
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLPFYTPD
DITLNNSVALNPIDISIELNKAKSDLEESKEWIRRSNQKLDSIEDKIEEILSKIYHIENEIAR
IKKLIGEAEPEA
SEQ ID NO: 55 PIV200288
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 56 PIV200289
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTLVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 57 PIV200290
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNPIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 58 PIV200291
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLLLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 59 PIV200292
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALDQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 60 PIV200293
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLPFYTPD
DITLNNSVALNPIDISIELNKAKSDLEESKEWIRRSNQKLDSIEDKIEEILSKIYHIENEIAR
IKKLIGEAEPEA
SEQ ID NO: 61 PIV200294
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTLVTSDIVPRYAF
VNGGVVANCITTTCTCNGIGNRINQPPDQGVKIITHKECNTIGINGMLFNTNKEGTLPFYTPD
DITLNNSVALNPIDISIELNKAKSDLEESKEWIRRSNQKLDSIEDKIEEILSKIYHIENEIAR
IKKLIGEAEPEA
SEQ ID NO: 62 PIV200295
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLPFYTPD
DITLNNSVALNPIDISIELNKAKSDLEESKEWIRRSNQKLDSIEDKIEEILSKIYHIENEIAR
IKKLIGEAEPEA
SEQ ID NO: 63 PIV200296
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLLLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLPFYTPD
DITLNNSVALNPIDISIELNKAKSDLEESKEWIRRSNQKLDSIEDKIEEILSKIYHIENEIAR
IKKLIGEAEPEA
SEQ ID NO: 64 PIV200297
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALDQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLPFYTPD
DITLNNSVALNPIDISIELNKAKSDLEESKEWIRRSNQKLDSIEDKIEEILSKIYHIENEIAR
IKKLIGEAEPEA
SEQ ID NO: 65 PIV200298
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTLVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 66 PIV200299
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 67 PIV200300
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLLLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 68 PIV200301
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALDQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 69 PIV200302
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTLVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 70 PIV200303
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLLLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTLVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 71 PIV200304
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALDQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTLVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 72 PIV200305
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 73 PIV200306
MPISILLIITTMIMASHCQIDITKLQHVGVLVNSPKGCKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNCQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 74 PIV200307
MPISILLIITTMIMASHCQIDITKLQHVGVLVNSPKGMKISQNFETRYLILSLIPKIEDSNSC
GDQQIKQYKRLLDRLIIPLYDCLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSCIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 75 PIV200308
MPISILLIITTMIMASHCQIDITKLQHVGVLVNSPKGMKISQNFETRYLILSLIPKIEDSNSC
GDQQIKQYKRLLDRLIIPLYDGLRLMKDVIVINQESNENTDPRTERFFGGVIGTIALGVATSA
QITAAVALVEAKQARSDIEKLKEAIRDTNKAVQSVQSSVGPLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 76 PIV200309
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 77 PIV200310
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLPFYTPD
DITLNNSVALNPIDISIELNKAKSDLEESKEWIRRSNQKLDSIEDKIEEILSKIYHIENEIAR
IKKLIGEAEPEA
SEQ ID NO: 78 PIV200311
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 79 PIV200312
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTLVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 80 PIV200313
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSPGNLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 81 PIV200314
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 82 PIV200315
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 83 PIV200316
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 84 PIV200317
MPISILLIITTMIMASHCQIDITKLQHVGVLVNSPKGCKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNCQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 85 PIV200318
MPISILLIITTMIMASHCQIDITKLQHVGVLVNSPKGMKISQNFETRYLILSLIPKIEDSNSC
GDQQIKQYKRLLDRLIIPLYDCLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPCIAR
LGCEACGLQLGIALTQHYSELTNIFGDNIGSCQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 86 PIV200319
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGPLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 87 PIV200320
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 88 PIV200321
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLPFYTPD
DITLNNSVALNPIDISIELNKAKSDLEESKEWIRRSNQKLDSIEDKIEEILSKIYHIENEIAR
IKKLIGEAEPEA
SEQ ID NO: 89 PIV200322
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 90 PIV200323
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTLVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 91 PIV200324
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPCIAR
LGCEACGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 92 PIV200325
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 93 PIV200326
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPCIAR
LGCEACGLLLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 94 PIV200327
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPCIAR
LGCEACGLQLGIALDQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 95 PIV200328
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPSIAR
LGCEAAGLLLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 96 PIV200329
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPSIAR
LGCEAAGLQLGIALDQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 97 PIV200330
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLLLGIALDQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 98 PIV200721
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 99 PIV200722
MPISILLIITTMIMASHCQIDITKLQHVGVLVNSPKGMKISQNFETRYLILSLIPKIEDSNSC
GDQQIKQYKRLLDRLIIPLYDGLRLQKDVIVINQESNENTDPRTERFFGGVIGTIALGVATSA
QITAAVALVEAKQARSDIEKLKEAIRDTNKAVQSVQSSPGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 100 PIV200723
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLLLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 101 PIV200724
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALDQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 102 PIV200878
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 103 PIV200879
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 104 PIV200880
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 105 PIV200881
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 106 PIV200882
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 107 PIV200883
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 108 PIV200884
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 109 PIV200885
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 110 PIV200886
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 111 PIV200887
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 112 PIV200888
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 113 PIV200889
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 114 PIV200890
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 115 PIV200891
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 116 PIV200892
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 117 PIV200893
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 118 PIV200894
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 119 PIV200895
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 120 PIV200896
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 121 PIV200897
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 122 PIV200898
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 123 PIV200899
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 124 PIV200900
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 125 PIV200901
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 126 PIV200902
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 127 PIV200903
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 128 PIV200904
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 129 PIV200905
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 130 PIV200906
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 131 PIV200907
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 132 PIV200908
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 133 PIV200909
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 134 PIV200910
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 135 PIV200911
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 136 PIV200912
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 137 PIV200913
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 138 PIV200914
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 139 PIV200915
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 140 PIV200916
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 141 PIV200917
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 142 PIV200918
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 143 PIV200919
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 144 PIV200920
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 145 PIV200921
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 146 PIV200922
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 147 PIV200923
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 148 PIV200924
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 149 PIV200925
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 150 PIV200926
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 151 PIV200927
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 152 PIV200928
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 153 PIV200929
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 154 PIV200930
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLLLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 155 PIV200931
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 156 PIV200932
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 157 PIV200933
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 158 PIV200934
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 159 PIV200935
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 160 PIV200936
MPISILLIITTMIMASHCQIDITKLQHVGVLVNSPKGMKIPQNFETRYLILSLIPKIE
DSNSCGDQQIKQYKRLLDRLIIPLYDGLRLMKDVIVTNQESNENTDPRTERFFGGVIGTIALG
VATSAQITAAVALVEAKQARSDIEKLKEAIRDTNKAVQSVQSSVGPPIVAIKSVQDYVNKEIV
PCIARLGCEACGLLLGIALTQHYSELTNIFGDNIGSLIEKGIKLQGIASLYRTNITEIFTTST
VDKYDIYDLLFTESIKVRVIDVDLNDYSITLQVRLPLLTRLLNTQIYKVDSISYNIQNREWYI
PLPSHIMTKGAFLGGADVKECIEAPSSYICPSDPGFVLNHEMESCLSGNISQCPRTTVTSDIV
PRYAFVNGGVVANCITTTCTCNGIGNRINQPPDQGVKIITHKECNTIGINGMLFNTNKEGTLA
FYTPDDITLNNSVALNPIDISIELNKAKSDLEESKEWIRRSNQKLDSIEDKIEEILSKIYHIE
NEIARIKKLIGEAEPEA
SEQ ID NO: 161 PIV200937
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLQLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 162 PIV200938
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPCIAR
LGCEACGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 163 PIV200939
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 164 PIV200940
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPCIAR
LGCEACGLLLGIALDQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 165 PIV200941
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIGSGEPEA
SEQ ID NO: 166 PIV200943
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWIRRSNQKLDSIGSGEPEA
SEQ ID NO: 167 PIV200945
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRVNQKLDSIGSGEPEA
SEQ ID NO: 168 PIV200947
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKVKSDLEEVKEWIRRSNQKLDSIGSGEPEA
SEQ ID NO: 169 PIV200949
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKVKSDLEESKEWIRRVNQKLDSIGSGEPEA
SEQ ID NO: 170 PIV200950
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWLRRSNQKLDSIGSGEPEA
SEQ ID NO: 171 PIV200951
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWIRRVNQKLDSIGSGEPEA
SEQ ID NO: 172 PIV200952
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWLRRVNQKLDSIGSGEPEA
SEQ ID NO: 173 PIV200953
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKVKSDLEEVKEWLRRSNQKLDSIGSGEPEA
SEQ ID NO: 174 PIV200954
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKVKSDLEEVKEWIRRVNQKLDSIGSGEPEA
SEQ ID NO: 175 PIV200955
MPISILLIITTMIMASHCQIDITKLQHVGVLVNSPKGMKISQNFETRYLILSLIPKIEDSNSC
GDQQIKQYKRLLDRLIIPLYDGLRLMKDVIVINQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKVKSDLEESKEWLRRVNQKLDSIGSGEPEA
SEQ ID NO: 176 PIV200956
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWLRRVNQKLDSIGSGEPEA
SEQ ID NO: 177 PIV200957
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKVKSDLEEVKEWLRRVNQKLDSIGSGEPEA
SEQ ID NO: 178 PIV200958
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKVKSDFEEVKEWLRRVNQKLDSIGSGEPEA
SEQ ID NO: 179 PIV200960
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRSNQKLDSIGGSEPEA
SEQ ID NO: 180 PIV200962
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRVNQKLDSIGGSEPEA
SEQ ID NO: 181 PIV200964
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKVKSDLEEVKEWLRRVNQKLDSIGGSEPEA
SEQ ID NO: 182 PIV200965
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKVKSDFEEVKEWLRRVNQKLDSIGGSEPEA
SEQ ID NO: 183 PIV200967
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWIRRSNQKLDSIGSGEPEA
SEQ ID NO: 184 PIV200969
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRVNQKLDSIGSGEPEA
SEQ ID NO: 185 PIV200971
MPISILLIITTMIMASHCQIDITKLQHVGVLVNSPKGMKISQNFETRYLILSLIPKIE
DSNSCGDQQIKQYKRLLDRLIIPLYDGLRLQKDVIVTNQESNENTDPRTERFFGGVIGTIALG
VATSAQITAAVALVEAKQARSDIEKLKEAIRDTNKAVQSVQSSPGNLIVAIKSVQDYVNKEIV
PCIARLGCEACGLQLGIALTQHYSELTNIFGDNIGSLQEKGIKLQGIASLYRTNITEIFTTST
VDKYDIYDLLFTESIKVRVIDVDLNDYSITLQVRLPLLTRLLNTQIYKVDSISYNIQNREWYI
PLPSHIMTKGAFLGGADVKECIEAFSSYICPSDPGFVLNHEMESCLSGNISQCPRTTVTSDIV
PRYAFVNGGVVANCITTTCTCNGIGNRINQPPDQGVKIITHKECNTIGINGMLFNTNKEGTLA
FYTPDDITLNNSVALNPIDISIELNKVKSDLEEVKEWIRRSNQKLDSIGSGEPEA
SEQ ID NO: 186 PIV200973
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKVKSDLEESKEWIRRVNQKLDSIGSGEPEA
SEQ ID NO: 187 PIV200974
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWLRRSNQKLDSIGSGEPEA
SEQ ID NO: 188 PIV200975
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWIRRVNQKLDSIGSGEPEA
SEQ ID NO: 189 PIV200976
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWLRRVNQKLDSIGSGEPEA
SEQ ID NO: 190 PIV200977
MPISILLIITTMIMASHCQIDITKLQHVGVLVNSPKGMKISQNFETRYLILSLIPKIE
DSNSCGDQQIKQYKRLLDRLIIPLYDGLRLQKDVIVTNQESNENTDPRTERFFGGVIGTIALG
VATSAQITAAVALVEAKQARSDIEKLKEAIRDTNKAVQSVQSSPGNLIVAIKSVQDYVNKEIV
PCIARLGCEACGLQLGIALTQHYSELTNIFGDNIGSLQEKGIKLQGIASLYRTNITEIFTTST
VDKYDIYDLLFTESIKVRVIDVDLNDYSITLQVRLPLLTRLLNTQIYKVDSISYNIQNREWYI
PLPSHIMTKGAFLGGADVKECIEAFSSYICPSDPGFVLNHEMESCLSGNISQCPRTTVTSDIV
PRYAFVNGGVVANCITTTCTCNGIGNRINQPPDQGVKIITHKECNTIGINGMLFNTNKEGTLA
FYTPDDITLNNSVALNPIDISIELNKVKSDLEEVKEWLRRSNQKLDSIGSGEPEA
SEQ ID NO: 191 PIV200978
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKVKSDLEEVKEWIRRVNQKLDSIGSGEPEA
SEQ ID NO: 192 PIV200979
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKVKSDLEESKEWLRRVNQKLDSIGSGEPEA
SEQ ID NO: 193 PIV200980
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWLRRVNQKLDSIGSGEPEA
SEQ ID NO: 194 PIV200982
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSPGNLIVAIKSVQDYVNKEIVPCIAR
LGCEACGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKVKSDFEEVKEWLRRVNQKLDSIGSGEPEA
SEQ ID NO: 195 PIV201082
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIEDKIEEILSKIYHIENEIAR
IKKLIGEAEPEA
SEQ ID NO: 196: PIV201105
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWIRRVNQKLDSIGSGEPEA
SEQ ID NO: 197 PIV201106
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWIRRVNQKLDSIGSGEPEA
SEQ ID NO: 198 PIV201107
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWIRRVNQKLDSIGSGEPEA
SEQ ID NO: 199 PIV201108
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWIRRVNQKLDSIGSGEPEA
SEQ ID NO: 200 PIV201109
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWIRRVNQKLDSIGSGEPEA
SEQ ID NO: 201 PIV201110
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIGSGEPEA
SEQ ID NO: 202 PIV201111
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRVNQKLDSIGSGEPEA
SEQ ID NO: 203 PIV201112
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWIRRSNQKLDSIGSGEPEA
SEQ ID NO: 204 PIV201113
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIGSGEPEA
SEQ ID NO: 205 PIV201114
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWIRRVNQKLDSIGSGEPEA
SEQ ID NO: 206 PIV201115
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 207 PIV201116
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEESKEWIRRSNQKLDSIEDKIEEILSKIYHIENEIAR
IKKLIGEAEPEA
SEQ ID NO: 208 PIV201254
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIGSGEPEA
SEQ ID NO: 209 PIV201255
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIGSGEPEA
SEQ ID NO: 210 PIV201256
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGPPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIGSGEPEA
SEQ ID NO: 211 PIV201257
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAPSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALNPIDISIELNKAKSDLEEVKEWIRRVNQKLDSIGSGEPEA
SEQ ID NO: 212 PIV210233
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIGSGEPEA
SEQ ID NO: 213 PIV210234
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIGSGEPEA
SEQ ID NO: 214 PIV210235
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIGSGEPEA
SEQ ID NO: 215 PIV210236
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRVNQKLDSIGSGEPEA
SEQ ID NO: 216 PIV210237
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRSNQKLDSIGSGEPEA
SEQ ID NO: 217 PIV210238
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGSGEPEA
SEQ ID NO: 218 PIV210294
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEvKEWIRRvNQKLDSIGGSEPEA
SEQ ID NO: 219 PIV210295
MPISILLIITTMIMASHCQIDITKLQHVGVLVNSPKGMKIpQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGGSEPEA
SEQ ID NO: 220 PIV210296
MPISILLIITTMIMASHCQIDITKLQHVGVLVNSPKGMKIpQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEvKEWIRRvNQKLDSIGGSEPEA
SEQ ID NO: 221 PIV210297
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNpIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGGSEPEA
SEQ ID NO: 222 PIV210298
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNpIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEvKEWIRRvNQKLDSIGGSEPEA
SEQ ID NO: 223 PIV210299
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGpLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGGSEPEA
SEQ ID NO: 224 PIV210300
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGpLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEvKEWIRRvNQKLDSIGGSEPEA
SEQ ID NO: 225 PIV210301
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEApSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGGSEPEA
SEQ ID NO: 226 PIV210302
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEApSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEvKEWIRRvNQKLDSIGGSEPEA
SEQ ID NO: 227 PIV210303
MPISILLIITTMIMASHCQIDITKLQHVGVLVNSPKGMKISQNFETRYLILSLIPKIEDSNSC
GDQQIKQYKRLLDRLIIPLYDGLRLmKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGGSEPEA
SEQ ID NO: 228 PIV210304
MPISILLIITTMIMASHCQIDITKLQHVGVLVNSPKGMKISQNFETRYLILSLIPKIEDSNSC
GDQQIKQYKRLLDRLIIPLYDGLRLmKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEvKEWIRRvNQKLDSIGGSEPEA
SEQ ID NO: 229 PIV210305
MPISILLIITTMIMASHCQIDITKLQHVGVLVNSPKGMKIpQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNpIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGGSEPEA
SEQ ID NO: 230 PIV210306
MPISILLIITTMIMASHCQIDITKLQHVGVLVNSPKGMKIpQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNpIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEvKEWIRRvNQKLDSIGGSEPEA
SEQ ID NO: 231 PIV210307
MPISILLIITTMIMASHCQIDITKLQHVGVLVNSPKGMKISQNFETRYLILSLIPKIEDSNSC
GDQQIKQYKRLLDRLIIPLYDGLRLmKDVIVTNQESNENTDPRTERFFGGVIGTIALGVATSA
QITAAVALVEAKQARSDIEKLKEAIRDTNKAVQSVQSSVGNpIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGGSEPEA
SEQ ID NO: 232 PIV210308
MPISILLIITTMIMASHCQIDITKLQHVGVLVNSPKGMKISQNFETRYLILSLIPKIEDSNSC
GDQQIKQYKRLLDRLIIPLYDGLRLmKDVIVTNQESNENTDPRTERFFGGVIGTIALGVATSA
QITAAVALVEAKQARSDIEKLKEAIRDTNKAVQSVQSSVGNpIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEvKEWIRRvNQKLDSIGGSEPEA
SEQ ID NO: 233 PIV210309
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSpGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGGSEPEA
SEQ ID NO: 234 PIV210310
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSpGNLIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEvKEWIRRvNQKLDSIGGSEPEA
SEQ ID NO: 235 PIV210311
MPISILLIITTMIMASHCQIDITKLQHVGVLVNSPKGMKISQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLILGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEESKEWIRRSNQKLDSIGGSEPEA
SEQ ID NO: 236 PIV210312
MPISILLIITTMIMASHCQIDITKLQHVGVLVNSPKGMKISQNFETRYLILSLIPKIE
DSNSCGDQQIKQYKRLLDRLIIPLYDGLRLQKDVIVTNQESNENTDPRTERFFGGVIGTIALG
VATSAQITAAVALVEAKQARSDIEKLKEAIRDTNKAVQSVQSSVGNLIVAIKSVQDYVNKEIV
PSIARLGCEAAGLILGIALTQHYSELTNIFGDNIGSLQEKGIKLQGIASLYRTNITEIFTTST
VDKYDIYDLLFTESIKVRVIDVDLNDYSITLQVRLPLLTRLLNTQIYKVDSISYNIQNREWYI
PLPSHIMTKGAFLGGADVKECIEAFSSYICPSDPGFVLNHEMESCLSGNISQCPRTTVTSDIV
PRYAFVNGGVVANCITTTCTCNGIGNRINQPPDQGVKIITHKECNTIGINGMLFNTNKEGTLA
FYTPDDITLNNSVALDPIDISIELNKAKSDLEEvKEWIRRvNQKLDSIGGSEPEA
SEQ ID NO: 237 PIV211368
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIG
SEQ ID NO: 238 PIV211886
MPISILLIITTMIMASHCQIDITKLQHVGVLVNSPKGMKISQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIG
SEQ ID NO: 239 PIV211887
MPISILLIITTMIMASHCQIDITKLQHVGVLVNSPKGMKIPQNFETRYLILSLIPKIEDSNSC
GDQQIKQYKRLLDRLIIPLYDGLRLQKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLQEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIG
SEQ ID NO: 241 PIV211889
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRSNQKLDSIG
SEQ ID NO: 242 PIV211890
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTERFFGGVIGTIALGVATSA
QITAAVALVEAKQARSDIEKLKEAIRDTNKAVQSVQSSVGNLIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIG
SEQ ID NO: 243 PIV220922
MPISILLIITTMIMASHCQIDITKLQHVGVLVNSPKGMKIPQNFETRYLILSLIPKIEDSNSC
GDQQIKQYKRLLDRLIIPLYDGLRLMKDVIVaNQESNENTDPRTEQFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIG
SEQ ID NO: 244 PIV220923
MPISILLIITTMIMASHCQIDITKLQHVGVLVNSPKGMKIPQNFETRYLILSLIPKIdDSNSC
GDQQIKQYKRLLDRLIIPLYDGLRLMKDVIVaNQESNENTDPRTEQFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIG
SEQ ID NO: 245
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTEQFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIG
SEQ ID NO: 246
MPISILLIITTMIMASHCQIDITKLQHVGVLVNSPKGMKIPQNFETRYLILSLIPKIdDSNSC
GDQQIKQYKRLLDRLIIPLYDGLRLMKDVIVTNQESNENTDPRTEQFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPD
DITLNNSVALDPIDISIELNKAKSDLEEVKEWIRRVNQKLDSIG
SEQ ID NO: 247
MPISILLIITTMIMASHCQIDITKLQHVGVLVNSPKGMKIPQNFETRYLILSLIPKIEDSNSC
GDQQIKQYKRLLDRLIIPLYDGLRLMKDVIVaNQESNENTDPRTEQFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPA
DITLNNSVALDPIDISIELNKAKSDLEEVKEWIRRVNQKLDSIG
SEQ ID NO: 248
MPISILLIITTMIMASHCQIDITKLQHVGVLVNSPKGMKIPQNFETRYLILSLIPKIdDSNSC
GDQQIKQYKRLLDRLIIPLYDGLRLMKDVIVaNQESNENTDPRTEQFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPA
DITLNNSVALDPIDISIELNKAKSDLEEVKEWIRRVNQKLDSIG
SEQ ID NO: 249
MPISILLIITTMIMASHCQIDITKLQHVGVLVNSPKGMKIPQNFETRYLILSLIPKIEDSNSC
GDQQIKQYKRLLDRLIIPLYDGLRLMKDVIVTNQESNENTDPRTEQFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPA
DITLNNSVALDPIDISIELNKAKSDLEEVKEWIRRVNQKLDSIG
SEQ ID NO: 250
MPISILLIITTMIMASHCQIDITKLQHVGVLVNSPKGMKIPQNFETRYLILSLIPKIdDSNSC
GDQQIKQYKRLLDRLIIPLYDGLRLMKDVIVTNQESNENTDPRTEQFFGGVIGTIALGVATSA
QITAAVALVEAKQARSDIEKLKEAIRDTNKAVQSVQSSVGNPIVAIKSVQDYVNKEIVPSIAR
LGCEAAGLQLGIALTQHYSELTNIFGDNIGSLIEKGIKLQGIASLYRTNITEIFTTSTVDKYD
IYDLLFTESIKVRVIDVDLNDYSITLQVRLPLLTRLLNTQIYKVDSISYNIQNREWYIPLPSH
IMTKGAFLGGADVKECIEAFSSYICPSDPGFVLNHEMESCLSGNISQCPRTTVTSDIVPRYAF
VNGGVVANCITTTCTCNGIGNRINQPPDQGVKIITHKECNTIGINGMLFNTNKEGTLAFYTPA
DITLNNSVALDPIDISIELNKAKSDLEEVKEWIRRVNQKLDSIG

1. A stabilized pre-fusion human parainfluenza virus 3 (HPIV3) F protein, comprising an F1 and an F2 domain comprising an amino acid sequence of the F1 and F2 domain of an F protein of an HPIV3 strain, wherein the amino acid residue at position 41 is P, and the amino acid residue at position 89 is M and the amino acid residue at position 222 is I, and the amino acid residue at position 168 is P, and the amino acid residue at position 470 is V and the amino acid residue at position 477 is V, and the amino acid residue at position 109 is Q, wherein the numbering of the amino acid positions is according to the numbering of amino acid residues in SEQ ID NO: 1.

2. The protein according to claim 1, wherein furthermore the amino acid residue at position 95 is A, and/or the amino acid residue at position 441 is A, and/or the amino acid residue at position 58 is D.

3. The protein according to claim 1, comprising a truncated F1 domain.

4. The protein according to claim 3, wherein the truncated F1 domain does not comprise the transmembrane and cytoplasmic regions.

5. The protein according to claim 3, wherein the truncated F1 domain comprises the amino acids 110-484, preferably the amino acids 110-485 of the HPIV3 F protein.

6. The protein according to claim 3, wherein a heterologous trimerization domain is linked to the truncated F1 domain.

7. The protein according to claim 1, comprising an amino acid sequence selected from the group consisting of SEQ ID NO: 243-250, or a fragment thereof, preferably comprising the amino acid sequence of SEQ ID NO: 243 or a fragment thereof.

8. A nucleic acid molecule encoding a protein according to claim 1.

9. The nucleic acid molecule according to claim 8, wherein the nucleic acid molecule is DNA or RNA.

10. The nucleic acid molecule according to claim 9, wherein the RNA is mRNA, modified mRNA, self-replicating RNA, or circular mRNA.

11. A nucleic acid molecule according to claim 8, encoding a protein comprising an amino acid sequence selected from the group consisting of SEQ ID NO: 243-250, or a fragment thereof, preferably comprising the amino acid sequence of SEQ ID NO: 243 or a fragment thereof.

12. A vector comprising a nucleic acid molecule according to claim 8.

13. The vector according to claim 12, wherein the vector is a human recombinant adenoviral vector.

14. The vector according to claim 13, wherein the adenoviral vector is a replication-incompetent Ad26 adenoviral vector having a deletion of the E1 region and the E3 region.

15. A composition comprising a protein according to claim 1.

16. A method for vaccinating a subject against PIV3, the method comprising administering to the subject a composition according to claim 15.

17. A method for preventing infection and/or replication of PIV3 in a subject, comprising administering to the subject a vaccine according to claim 15.

18. A composition comprising a nucleic acid molecule according to claim 8.

19. A composition comprising a vector according to claim 12.
